(19) United States
(12) Patent Application Publication
(10) Pub. No.: US 2023/0250142 A1
(43) Pub. Date: Aug. 10, 2023

(54) DOCK TAG SYSTEM

(71) Applicant: GLAXOSMITHKLINE BIOLOGICALS SA, Rixensart (BE)

(72) Inventors: Matthew James BOTTOMLEY, Rockville, MD (US); Roberta COZZI, Siena (IT); Newton Muchugu WAHOME, Rockville, MD (US)

(73) Assignee: GLAXOSMITHKLINE BIOLOGICALS SA, Rixensart (BE)

(21) Appl. No.: 18/008,771

(22) PCT Filed: Jun. 11, 2021
(86) PCT No.: PCT/IB2021/055157

§ 371 Date: Dec. 7, 2022

Related U.S. Application Data
(60) Provisional application No. 63/038,237, filed on Jun. 12, 2020.

(51) Int. Cl. C07K 14/47; A61K 47/42; A61K 47/64; A61K 47/61; C12N 15/63

(52) U.S. Cl. CPC C07K 14/47; A61K 47/42; A61K 47/646; A61K 47/61; C12N 15/63; C07K 2319/33

(57) ABSTRACT

Pairs of peptides capable of forming spontaneous covalent bonds, and their uses, such as in forming fusion proteins.

### SEQUENCE LISTING

The instant application contains a Sequence Listing which has been submitted electronically in ASCII format and is hereby incorporated by reference in its entirety. Said ASCII copy, created on May 21, 2021, is named VU66933_WO_SL.txt and is 122,688 bytes in size.

### FIELD OF THE INVENTION

This invention relates to pairs of peptides capable of forming spontaneous covalent bonds, and their uses, such as in forming fusion proteins.

### BACKGROUND

Systems that allow spontaneous, irreversible bond formation between polypeptide pairs via isopeptide bonds have been described (see e.g., Zakeri et al., PNAS 109(12):E690-E697 (March 2012); Hatlem et al., Int. J. Mol Sciences 20 (2019)). The “SpyCatcher-SpyTag” is based on a modified domain from a Streptococcus pyogenes surface protein (SpyCatcher), which forms a covalent isopeptide bond with a cognate 13-amino-acid peptide (SpyTag); this system has been used in methods of protein ligation (WO 2011/098772). A similar system has been developed based on the pilus adhesin RrgA of S. pneumoniae (see, e.g., Veggiani et al., PNAS USA 2016; 113:1202-1207; Izore et al., Structure 2010; 18:106-115).

There is a need for additional polypeptide systems capable of spontaneously forming irreversible bonds with each other, for use in methods of protein ligation and fusion protein production.

### SUMMARY OF THE INVENTION

The inventors have designed peptide binding pairs that react specifically and spontaneously to form a stable covalent bond; such peptide pairs are useful as a ‘tag/dock’ system to join together heterologous polypeptides. As the peptide binding pairs are derived from Group B Streptococcus (Streptococcus agalactiae) polypeptides, they are termed herein GalacDock and GalacTag polypeptides.

One aspect of the present invention are isolated polypeptides that are GalacDock or GalacTag peptides.

A further aspect of the present invention are fusion proteins of a GalacDock or a GalacTag peptide, and a heterologous polypeptide.

A further aspect of the present invention is a fusion protein of a GalacTag peptide and a polypeptide subunit of a self-assembling protein nanoparticle, and nanoparticles made of such fusion proteins.

A further aspect of the present invention are GalacTag and GalacDock binding partner pairs, wherein when contacted with each other under suitable conditions, the GalacTag and GalacDock peptides bind to each other and form an isopeptide bond.

A further aspect of the present invention is a kit containing a GalacDock and a GalacTag binding partner pair, where the peptides are not covalently joined.

A further aspect of the present invention are nucleic acid molecules encoding polypeptides and fusion proteins of the invention; vectors of such nucleic acid molecules; and host cells containing such vectors.

A further aspect of the present invention are methods of producing the polypeptides, fusion proteins, and nanoparticles of the invention.

A further aspect of the present invention is a method of producing fusion proteins, by providing a GalacDock and GalacTag binding pair, where one member of the pair is a fusion protein with a heterologous molecule, and contacting the binding pair peptides under conditions that allow the formation of an isopeptide bond between the GalacTag and GalacDock peptides.

A further aspect of the present invention is a method of producing a protein nanoparticle that displays a heterologous molecule on its exterior surface, by providing a plurality or multiplicity of fusion proteins of a GalacTag peptide conjugated at the C-terminus to a NP polypeptide subunit, allowing self-assembly of NPs to provide NPs displaying GalacTags on the external surface of the NP, providing GalacDock binding partners, where the GalacDock peptide is conjugated to a heterologous molecule, and contacting the NPs and GalacDock-heterologous molecule conjugates under conditions that allow the formation of isopeptide bonds between the GalacTag and GalacDock peptides.

A further aspect of the present invention is pharmaceutical compositions comprising NPs, where heterologous molecules are displayed on the exterior NP surface using a GalacTag/GalacDock binding pair.

A further aspect of the present invention is the use of polypeptides, fusion proteins, or NPs of the present invention in pharmaceutical compositions.

A further aspect of the present invention is the use of NPs or pharmaceutical compositions of the present invention to induce an immune response, or in the treatment or prevention of disease or infection.

### BRIEF DESCRIPTION OF THE FIGURES

FIG. 1A-C Design of GalacTag/GalacDock polypeptides Minimum designed peptide length from the C-terminus of GBS BP pilus proteins for isopeptide formation, shown as a double dashed line; double solid line indicates extended length to modulate solubility and stability. BP-1 (FIG. 1A), BP-2a (FIG. 1B), and BP-2b (FIG. 1C).

FIG. 2A-B Depiction of a GalacTag covalently linked to Factor H Binding protein from N. meningitidis (fHbp var1.1), and attached via isopeptide bond to its corresponding GalacDock polypeptide, shown as a surface view (FIG. 2A) and a schematic view (FIG. 2B).

FIG. 3A SDS-PAGE electrophoresis results demonstrating that certain point mutations abolish isopeptide formation. Lane 3 demonstrates complex formation for the wildtype complex, evidenced by the band appearing at approx. MW 100 kDa, which corresponds to the expected MW for a heterodimer formed by Tag_6/fHbp and Dock_6. A higher-order molecular weight complex was not formed when variant GalacDock6 polypeptides were paired with ‘wildtype’ (wt) GalacTag6_fHbp (SEQ ID NO:20), or when variant GalacTag6_fHbp (N636A) was paired with ‘wild type’ (wt) GalacDoc6 (SEQ ID NO:6).

FIG. 3B Western blot with anti-fHbp antibody, 4B3, shows that the higher-order species formed when GalacDock6 and GalacTag6_fHbp were paired contains fHbp. Purified fHbp protein was used as a positive control (Lane 4).

FIG. 4 Shows the detection by SDS-PAGE electrophoresis and HPLC-SEC of GalacDock6 and GalacTag6_GBSFerritinLMG14747 complex formation.

FIG. 5A Shows the detection by SDS-PAGE electrophoresis and HPLC-SEC of GalacDock6 and GalacTag6_GBSFerritinDK-PW-092 complex formation.

FIG. 5B Shows the detection by SDS-PAGE electrophoresis of GalacDock6 and GalacTag6_GBSFerritinDK-PW-092 complex formation over time.

FIG. 6 Shows the detection by SDS-PAGE electrophoresis and HPLC-SEC of GalacDock6 and GalacTag6_GBSFerritinDK-PW-092+N-terminal helix.

FIG. 7A-7C Negative stain transmission electron microscopy (TEM) images of GalacDock6 and GalacTag6_GBSFerritin complexes: (A) TEM micrograph of GalacTag6_GBSFerritinLMG14747 complex, indicating a presence of aggregates in the preparation; (B) TEM micrograph of GalacTag6_GBSFerritinDK-PW-092+ helix nanoparticle complex; (C) TEM micrograph of GalacTag6_GBSFerritinDK-PW-092 complex, indicating a presence of formed nanoparticles, arrayed with visible GalacDock/GalacTag polypeptides (inset).

FIG. 7D Model of a GBS ferritin nanoparticle complexed with GBS GalacDock/GalacTag polypeptides formed via isopeptide covalent bond formation.

FIG. 8A-8C Thermostability Assessment of GalacDock6 and GalacTag6_GBSFerritin complex: (A) Differential scanning calorimetry (DSC) thermogram of GalacDock6, indicating the presence of three thermal unfolding transitions; (B) DSC thermogram of GalacTag6_GBSFerritinDK-PW-092 nanoparticle, indicating the presence of two thermal transitions; (C) DSC thermogram of GalacTag6_GBSFerritinDK-PW-092 nanoparticle in complex with GalacDock6, indicating the presence of three thermal unfolding transitions, with the apparent stabilization of Tm₁ (65.5° C.) relative to Tm₁ (45° C.) in uncomplexed GalacDock6, as shown in panel A. Models of proteins provided for guidance.

FIG. 9 Analytical ultracentrifugation (AUC) of GalacDock6, GalacTag6_GBSFerritinDK-PW-092 nanoparticle, and a nanoparticle complex of GalacDock6 with GalacTag6_GBSFerritinDK-PW-092. The sedimentation coefficient analysis indicates the presence of three distinct size distributions. Models of proteins provided for guidance.

FIG. 10A-E: illustrates the multiple domains of (A) GBS BP1, (B) GBS BP2a, (C) GBS BP2b, (D) GBS AP1 PI-1 (AP1-1), and (E) AP1 PI-2a (AP1-2a), where SP indicates Signal Peptide and TM indicates transmembrane domain (not to scale). Lysine (K) and asparagine (N) residues are indicated, numbering corresponding to reference sequences SEQ ID NO: 40, SEQ ID NO: 41, SEQ ID NO: 42, SEQ ID NO: 61 and SEQ ID NO: 62, respectively.

SEQUENCES

| SEQ | | |
|---|---|---|
| ID | Description | Length |
| 43 | GalacDock1: amino acids 2-259 of BP2b (SEQ ID NO: 26) | 258 |
| 1 | GalacDock1 with MHHHHHHS leader | 266 |
| 44 | GalacDock2: amino acids 2-267 of BP2b (SEQ ID NO: 26) | 266 |
| 2 | GalacDock2 with MHHHHHHS leader | 274 |
| 45 | GalacDock3: amino acids 2-274 of BP2b (SEQ ID NO: 26) | 273 |
| 3 | GalacDock3 with MHHHHHHS leader | 281 |
| 46 | GalacDock4: amino acids 1-291 of BP1 (SEQ ID NO: 27) | 291 |
| 4 | GalacDock4 with MHHHHHHS leader | 299 |
| 47 | GalacDock5: amino acids 1-308 of BP1 (SEQ ID NO: 27) | 308 |
| 5 | GalacDock5 with MHHHHHHS leader | 316 |
| 48 | GalacDock6: amino acids 1-419 of BP2a (SEQ ID NO: 28) | 419 |
| 6 | GalacDock6 with MHHHHHHS leader | 427 |
| 19 | GalacDock7: amino acids 1-442 of BP2a (SEQ ID NO: 28) | 422 |
| 7 | GalacDock7 with MHHHHHHS leader | 450 |
| 50 | GalacDock8: amino acids 744-857 of AP1-1 (SEQ ID NO: 61) | 114 |
| 51 | GalacDock9: amino acids 746-857 of AP1-2a (SEQ ID NO: 62) | 112 |
| | GalacTag | |
| 52 | GalacTag1: amino acids 261-287 of BP2b (SEQ ID NO: 26) | 27 |
| 8 | GalacTag1 with N-terminal Methionine (M) | 28 |
| 53 | GalacTag2: amino acids 271-287 of BP2b (SEQ ID NO: 26) | 18 |
| 9 | GalacTag2 with N-terminal Methionine (M) | 19 |
| 54 | GalacTag3: amino acids 278-287 of BP2b (SEQ ID NO: 26) | 12 |
| 10 | GalacTag3 with N-terminal Methionine-Alanine (MA) | 12 |
| 55 | GalacTag4: amino acids 294-319 of BP1 (SEQ ID NO: 27) | 28 |
| 11 | GalacTag4 with N-terminal Methionine-Alanine (MA) | 28 |
| 56 | GalacTag5: amino acids 312-319 of BP1 (SEQ ID NO: 27) | 9 |
| 12 | GalacTag5 with N-terminal Methionine-Alanine (MA) | 10 |
| 57 | GalacTag6: amino acids 422-452 of BP2a (SEQ ID NO: 28) | 33 |
| 13 | GalacTag6 with N-terminal Methionine-Alanine (MA) | m |
| 58 | GalacTag7: amino acids 445-452 of BP2a (SEQ ID NO: 28) | 10 |
| 14 | GalacTag7 with N-terminal Methionine-Alanine (MA) | 10 |
| 59 | GalacTag8: amino acids 732-743 of AP1-1 (SEQ ID NO: 61) | 12 |
| 60 | GalacTag9: amino acids 730-745 of AP1-2a (SEQ ID NO: 62) | 16 |
| | GalacTag + fHbp fusions | |
| 15 | GalacTag1: (M) + (amino acids 261-287 of BP2b (SEQ ID NO: 26)) + (GGSGG | 285 |
| | linker) + (fHbp sequence (SEQ ID NO: 29)) + (6-His tag) | |
| 16 | GalacTag2: (M) + (amino acids 271-287 of BP2b (SEQ ID NO: 26)) + (GGSGG | 276 |
| | linker) + (fHbp sequence (SEQ ID NO: 29)) + (6-His tag) | |
| 17 | GalacTag3: (MA) + (amino acids 278-287 of BP2b (SEQ ID NO: 26)) + (GGSGG | 269 |
| | linker) + (fHbp sequence (SEQ ID NO: 29)) + (6-His tag) | |
| 18 | GalacTag4: (MA) + (amino acids 294-319 of BP1 (SEQ ID NO: 27)) + (GGSGG | 285 |
| | linker) + (fHbp sequence (SEQ ID NO: 29)) + (6-His tag) | |
| 19 | GalacTag5: (MA) + (amino acids 312-319 of BP1 (SEQ ID NO: 27)) + (GGSGG | 267 |
| | linker) + (fHbp sequence (SEQ ID NO: 29)) + (6-His tag) | |
| 20 | GalacTag6: (MA) + (amino acids 422-452 of BP2a (SEQ ID NO: 28)) + (GGSGG | 290 |
| | linker) + (fHbp sequence (SEQ ID NO: 29)) + (6-His tag) | |
| 21 | GalacTag7: (MA) + (amino acids 445-452 of BP2a (SEQ ID NO: 28)) + (GGSGG | 267 |
| | linker) + (fHbp sequence (SEQ ID NO: 29)) + (6-His tag) | |
| | GalacTag + GBS Ferritin NP subunit | |
| 22 | GalacTag6 + Ferritin NP Subunit: LMG_14747 | 199 |
| 23 | GalacTag6 + GBS ferritin NP subunit from DK-092-PW | 201 |
| 24 | GalacTag6 + GBS ferritin NP subunit from LMG 14747 w/N-terminal helix | 221 |
| 25 | GalacTag6 + GBS ferritin NP subunit from DK-092-PW w/N-terminal helix | 223 |
| | GBS pilin domains | |
| 26 | GBS pilin BP 2b-PDB: 4UZG_A. | 292 |
| 27 | GBS Pilin BP 1-PDB: 3PF2 | 319 |
| 28 | GBS pilin BP-2a-PDB: 2XTL | 452 |
| | fHbp used in GalacTag_fHbp constructs | |
| 29 | fHbp fragment used in GalacTag fHbp constructs | 246 |
| | GBS Ferritin subunit polypeptides | |
| 30 | GBS ferritin NP subunit from LMG 14747 | 153 |
| 31 | GBS ferritin NP subunit from DK-092-PW, with C124S substitution (compared to | 155 |
| | wildtype) | |
| 32 | GBS ferritin NP subunit from LMG 14747 w/N-terminal helix | 175 |
| 33 | GBS ferritin NP subunit from DK-092-PW w/N-terminal helix | 177 |
| | Tags and Linkers | |
| 34 | 6x His Tag (HHHHHH) | 6 |
| 35 | FLAG tag (Asp-Tyr-Lys-Asp-Asp-Asp-Asp-Lys) | 8 |
| 36 | Strep tag (Ala-Trp-Arg-His-Pro-Gln-Phe-Gly-Gly) | 9 |
| 37 | Tag: Trp-Ser-His-Pro-Gln-Phe-Glu-Lys | 8 |
| 38 | Trp-Ser-His-Pro-Gln-Phe-Glu-Lys-Gly-Gly-Gly-Ser-Gly- | 28 |
| | Gly-Gly-Ser-Gly-Gly-Gly-Ser-Trp-Ser-His-Pro-Gln-Phe-Glu-Lys | |
| 39 | GGSGG-linker used GalacTag and fHbp; and to connect GalacTag and | 5 |
| | GBS_Ferritin sequences | |
| 63 | GSGGG linker | 5 |
| | GBS BP Reference Sequences | |
| 40 | Reference sequence for BP-1 (GBS80) | 554 |
| 41 | Reference sequence for BP-2a (GBS59); | 675 |
| 42 | Reference sequence for BP-2b protein; isopeptide bonds in domain D2 (K187 | 502 |
| | and N330), and domain D3 (K358 and N462). | |
| 61 | Reference sequence for Ancillary protein 1 of PI-1 (AP1-1) protein | 890 |
| 62 | Reference sequence for Ancillary protein 1 of PI-2a (AP1-2a) protein | 901 |

### DETAILED DESCRIPTION

The present invention relates to pairs of peptides derived from Streptococcus agalactiae pilin protein, which pairs react specifically and spontaneously to form a stable covalent bond. The bond is stable under conditions (e.g., time, temperature, pH) that would result in the dissociation of non-covalent bonds. Such a polypeptide pair is referred to herein as a Tag/Dock system and, more specifically, GalacTag and GalacDock (from ‘agalactiae’). The peptide pairs may alternatively be described as binding partner proteins, or as a peptide tag and a binding partner. Covalent bonds may begin forming almost immediately after contacting a GalacTag and GalacDock binding pair under suitable conditions, e.g. within 15, 20, 25, 30, 40 or 45 minutes, or within 1, 2, 4, 8, 12, 16, 20 or 24 hours. The bond may form in phosphate-buffered saline (PBS) at pH 7.0 and at 25° C. Stability of a bond may be assessed by, for example, heating at 95° C. for 7 minutes in a solution containing 1% sodium dodecyl sulfate (SDS).

The present invention provides a method of joining two moieties, where one such moiety is bound to a GalacTag polypeptide, and the other is bound to a corresponding GalacDock polypeptide. The GalacTag polypeptide and GalacDock polypeptide are placed in contact with each other under conditions that enable the formation of an isopeptide bond between them. A moiety attached to a GalacDock or GalacTag polypeptide is referred to herein as a ‘target’ moiety (e.g., a target polypeptide), and include detectable labels, antigenic polypeptides, antigenic polysaccharides or oligosaccharides, and antigenic glycoconjugates. As used herein, neither GalacTag nor GalacDock polypeptides are considered target moieties.

The present invention further provides methods of joining two target moieties, by contacting (a) a first molecule comprising a first target moiety bound to a GalacTag polypeptide, and (b) a second molecule comprising a second target moiety bound to a GalacDock polypeptide, under conditions that allow the formation of an isopeptide bond between the GalacTag and GalacDock polypeptides.

Further aspects of the present invention are GalacTag and GalacDock recombinant proteins, recombinant fusion proteins comprising a GalacTag polypeptide and a target polypeptide, recombinant fusion proteins comprising a GalacDock polypeptide and a target polypeptide, GalacDock polypeptides conjugated to saccharide target moieties or glycoconjugate target moieties, nucleic acid molecules encoding said polypeptides and fusion proteins, vectors comprising said nucleic acid molecules, and host cells comprising said vectors or said nucleic acid molecules. Also provided by the present invention are GalacTag polypeptides conjugated to non-polypeptide target moieties, and GalacDock polypeptides conjugated to non-polypeptide target moieties.

A further aspect of the present invention is the use of a GalacTag/GalacDock binding pair to detect or purify a recombinant target moiety of interest, by recombinantly producing the target moiety as a fusion with one member of the binding pair and then detecting or purifying the target moiety by binding to the other member of the binding pair.

A further aspect of the present invention is nanoparticles (NP) which display, at the external surface, GalacTags. Such NPs may then be contacted with GalacDock polypeptides under conditions that enable the formation of an isopeptide bond between the GalacTag and GalacDock polypeptides. When the GalacDock peptide is bound to a target moiety, NPs displaying the target moiety on the external NP surface are produced.

A further aspect of the present invention is a method of producing NPs displaying one or more target moieties one the NP surface, the method comprising (a) expressing fusion polypeptides comprising or consisting of (i) a polypeptide subunit of a self-assembling NP and (ii) a GalacTag polypeptide, and allowing self-assembly of said NP subunits to provide an NP displaying GalacTag polypeptides on the external surface; and then (b) contacting said NP with GalacDock polypeptides bound to a target moiety, under conditions that allow the formation of an isopeptide bond between GalacTag and GalacDock.

A further aspect of the present invention is a kit comprising both members of a GalacDock/GalacTag binding pair. The GalacDock and/or GalacTag may be attached or conjugated to a target moiety (such as a heterologous antigenic molecule), a detectable label, a solid support (e.g., a plate or column), a polypeptide subunit of a self-assembling nanoparticle, or a multimeric protein nanoparticle.

### Group B Streptococcus and Pilus Islands

GalacTag and GalacDock polypeptides are derived from Streptococcus agalactiae (also known as “Group B Streptococcus” or “GBS”) pilus proteins, including Backbone Proteins (BP) and Ancillary Proteins (AP). GBS pili proteins are filamentous structures protruding from the bacterial surface which function in bacterial virulence and disease pathogenesis, and are composed of three structural proteins: the major pilus subunit (backbone protein, BP) that forms the pilus shaft and two ancillary proteins (AP1 and AP2). GBS expresses three structurally distinct pilus types, encoded by distinct genomic loci (pilus islands or PIs): PI-1, PI-2a, and PI-2b. Each pilus island consists of five genes that encode the pilus backbone protein (BP), the two ancillary proteins (AP1 and AP2); and two sortase proteins involved in the assembly of the pili. Each GBS strain carries at least one of the pilus islands. The sequences of the pilus structural proteins (BP, AP1 and AP2) encoded by these pilus islands are generally well conserved, although the sequence of the BP protein encoded by PI-2a (BP-2a) varies among GBS strains.

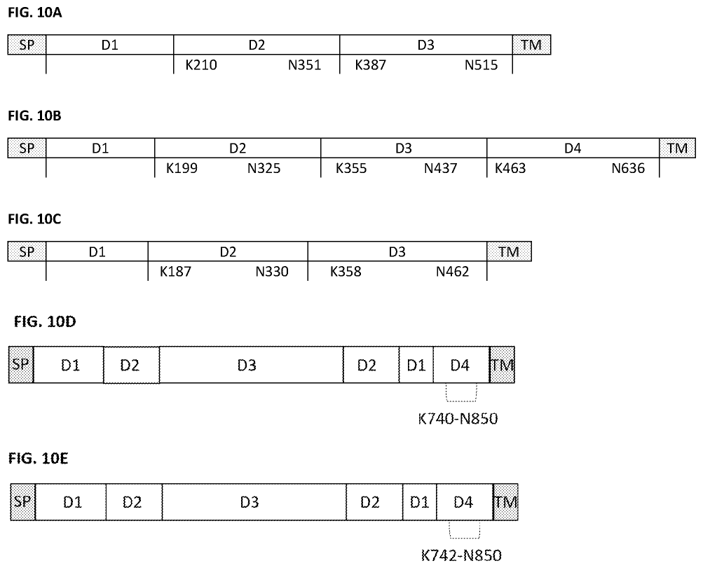

Each GBS pilus protein is composed of multiple domains (see FIG. 10A-E). The domains contain IgG-like secondary structure folds that are covalently stabilized by spontaneously formed isopeptide bonds. X-ray crystallography studies of domains D2-D3 (BP1, BP-2b), D2-D4 (BP-2a) and D4 (AP1-1 and AP1-2a) indicate the presence of stabilizing covalent isopeptide bonds.

### Tag/Capture Systems Based on Isopeptide Bonds:

The present inventors designed isopeptide bond-forming tag/dock systems based on sequences from GBS pilus BP and AP proteins. An isopeptide bond is a spontaneous auto-catalytic chemical event that covalently links asparagine (N) or aspartate (D) residues with a neighboring lysine (K) in the presence of a catalyzing glutamic acid (E). As discussed herein, a GalacTag/GalacDock binding pair is a GalacTag polypeptide having a contiguous sequence of at least five amino acids and no more than 50 amino acids from a GBS BP or AP protein, and comprising one or more residues involved in the isopeptide bond of that GBS BP or AP protein, and a GalacDock polypeptide having a contiguous sequence of at least 50 amino acids from a GBS BP or AP protein, and comprising residue(s) that will form an isopeptide bond with the GalacTag.

Combination with Therapeutic or Prophylactic Moieties

GalacDock and/or GalacTag binding partners may be conjugated to a compound which has a therapeutic or prophylactic effect, e.g., an antibiotic, antiviral, antigen, or antitumour agent. The GalacDock and/or GalacTag may additionally or alternatively be conjugated to a detectable label, for example a radiolabel, a fluorescent label, luminescent label, a chromophore label as well as to substances which generate a detectable substrate e.g. horse radish peroxidase, luciferase or alkaline phosphatase.

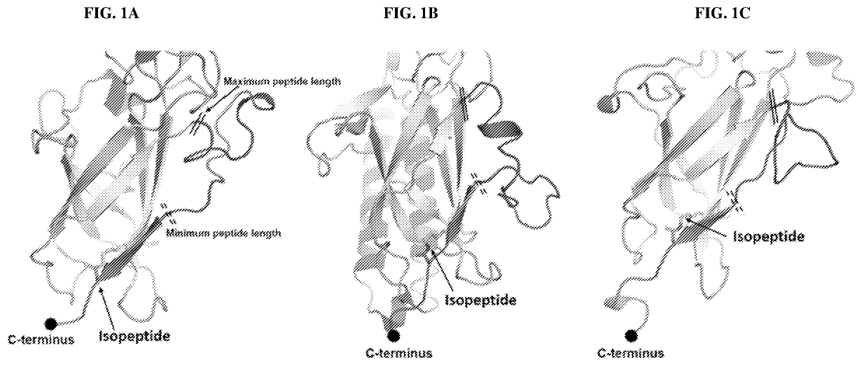

Accordingly, one embodiment of the present invention is fusion proteins comprising a GalacDock or GalacTag polypeptide and a heterologous antigenic polypeptide. The antigenic polypeptide may be covalently attached directly to the N-terminal or C-terminal amino acid of the GalacDock polypeptide, or covalently attached directly to the N-terminal or C-terminal amino acid of the GalacTag polypeptide (as long as the minimum peptide length is maintained, see FIG. 1), or a short (less than about 20, less than about 15, less than about 10, or less than about 5 amino acids) peptide linker sequence may be placed between the antigenic polypeptide and the GalacDock or GalacTag sequence.

In one embodiment, the GalacDock polypeptide is conjugated to polysaccharides or oligosaccharides, such as antigenic bacterial capsular polysaccharides, or immunogenic fragments thereof.

A further embodiment of the present invention provides recombinant polynucleotide sequences encoding such fusion proteins.

### Polypeptides

An amino acid or nucleic acid position (residue) in a polypeptide (or nucleotide) sequence may be identified by reference to the amino acid (nucleic acid) as numbered in a specified reference sequence Amino acid or nucleic acid positions in a sequence that are “comparable” or “corresponding” to those in a specified reference sequence can be determined by one of ordinary skill in the art using known information, and by sequence alignment using readily available and well-known alignment algorithms (such as BLAST, using default settings; ClustalW2, using default settings; or algorithm disclosed by Corpet, Nucleic Acids Research, 1998, 16(22):10881-10890, using default parameters). Orientation within a polypeptide is generally recited in an N-terminal to C-terminal direction, defined by the orientation of the amino and carboxy moieties of individual amino acids. Polypeptides are translated from the N-terminal or amino-terminus towards the C-terminal or carboxy-terminus.

Amino acid substitutions may be conservative substitutions Amino acids are commonly classified into distinct groups according to their side chains. For example, some side chains are considered non-polar, i.e. hydrophobic, while some others are considered polar, i.e. hydrophilic. Alanine (A), glycine (G), valine (V), leucine (L), isoleucine (I), methionine (M), proline (P), phenylalanine (F) and tryptophan (W) are considered to be hydrophobic amino acids, while serine (S), threonine (T), asparagine (N), glutamine (Q), tyrosine (Y), cysteine (C), lysine (K), arginine (R), histidine (H), aspartic acid (D) and glutamic acid (E) are considered to be polar amino acids. Regardless of their hydrophobicity, amino acids are also classified into subgroups based on common properties shared by their side chains. For example, phenylalanine, tryptophan and tyrosine are jointly classified as aromatic amino acids and will be considered as aromatic amino acids within the meaning of the present invention. Aspartate (D) and glutamate (E) are among the acidic or negatively charged amino acids, while lysine (K), arginine (R) and histidine (H) are among the basic or positively charged amino acids, and they will be considered as such in the sense of the present invention. Hydrophobicity scales are available which utilize the hydrophobic and hydrophilic properties of each of the 20 amino acids and allocate a hydrophobic score to each amino acid, creating thus a hydrophobicity ranking. As an illustrative example only, the Kyte and Dolittle scale may be used (Kyte et al. 1982. J. Mol. Bio. 157: 105-132). This scale allows one skilled in the art to calculate the average hydrophobicity within a segment of predetermined length.

The polypeptides of the present invention may contain an amino acid sequence known as a “tag” (distinct from a GalacTag) which facilitates purification (e.g. a polyhistidine-tag to allow purification on a nickel-chelating resin).

A “variant” of a polypeptide sequence includes amino acid sequences having one, two, three, or more amino acid substitutions, insertions and/or deletions when compared to the reference sequence. The variant may comprise an amino acid sequence which is at least 70%, at least 75%, at least 80%, at least 85%, at least 90%, at least 95%, at least 96%, at least 97%, at least 98%, or at least 99% identical to a full-length reference polypeptide.

The GalacTag and GalacDock polypeptides of the invention may be modified to introduce amino acid residues known in the art as capable of being chemically conjugated to a heterologous molecule. Alterations (amino acid substitutions, deletions, insertions) may be made to the GalacDock and GalacTag polypeptide sequences of the present invention, where said alterations do not affect the covalent bonding ability of the binding pair, or the ability to be conjugated to heterologous molecules, or the ability of the pair to form an isopeptide bond with each other when one or both of the Galac polypeptides are bound to a heterologous molecule.

According to the present invention, two polypeptides having a high degree of identity have amino acid sequences at least 80% identical, at least 85% identical, at least 87% identical, at least 90% identical, at least 92% identical, at least 94% identical, at least 96% identical, at least 98% identical or at least 99% identical. It will be understood by those of skill in the art that the similarity between two polypeptide sequences (or polynucleotide sequences), can be expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two polypeptide (or polynucleotide) sequences, the more similar are the higher order structures resulting from folding and assembly. Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Sequence identity between polypeptide sequences is preferably determined by pairwise alignment algorithm using the Needleman-Wunsch global alignment algorithm (Needleman and Wunsch, A General Method Applicable to the Search for Similarities in the Amino Acid Sequence of Two Proteins, 1970 J. Mol. Biol. 48(3): 443-453), using default parameters (e.g. with Gap opening penalty=10.0, and with Gap extension penalty=0.5, using the EBLOSUM62 scoring matrix). This algorithm is conveniently implemented in the needle tool in the EMBOSS package (Rice et al., EMBOSS: The European Molecular Biology Open Software Suite, 2000 Trends Genetics 16: 276-277). Sequence identity should be calculated over the entire length of the polypeptide sequence of the invention.

### Nanoparticles & Virus Like Particles

Virus-like particles and other protein nanoparticles (NPs) made of multiple self-assembling polypeptide subunits may be utilized in the present invention, to provide a scaffold to which one or more GalacTag and/or GalacDock moieties are attached. The NP can thus be considered a scaffold for displaying GalacTag and/or GalacDock(s) at the NP exterior surface. In one embodiment, the NP subunit polypeptide is linked to a GalacTag of the present invention prior to assembly of the NP, e.g., by use of a nucleotide sequence encoding a fusion protein of an NP subunit and a GalacTag sequence. Self-assembly of NP subunits places their N-terminals at the outer surface and C-terminals at the inner surface of the assembled NP. Fusion proteins of a GalacTag polypeptide and an NP subunit polypeptide, where the GalacTag is linked to the NP subunit N-terminus, will self-assemble into an NP displaying the GalacTag polypeptide on the exterior surface of the assembled NP. The NP can be considered a scaffold for displaying GalacTag polypeptides at the NP exterior surface. The number of GalacTags displayed at the NP surface will depend upon the number of subunits in the NP that are joined to GalacTags.

GalacTags may be linked directly at the N-terminus of an NP subunit sequence or attached thereto by a short amino acid linker sequence. Suitable polypeptide linkers include linkers of two or more amino acids. An illustrative polypeptide linker is one or more multimers of GGS or GSS, or variations thereof such as GGSGG (SEQ ID NO: 39) or GSGGG (SEQ ID NO: 63). Several (one to ten) N-terminal amino acid residues of the NP subunit polypeptide sequence may be deleted and replaced with the linker sequence.

One embodiment of the present invention is fusion proteins comprising an NP polypeptide subunit sequence and a GalacTag sequence attached N-terminally to the subunit sequence (directly or via a linker), and capable of self-assembly into an NP. “Self-assembly” of NPs refers to the oligomerization of polypeptide subunits into an ordered arrangement, driven by non-covalent interactions. Such noncovalent interactions may be any of electrostatic interactions, Π-interactions, van der Waals forces, hydrogen bonding, hydrophobic effects, or any combination thereof.

A GalacDock polypeptide can be covalently bound to a GalacTag displayed on the exterior of a NP, via the isopeptide binding of GalacDock/GalacTag system. The GalacDock may be attached to a target moiety; isopeptide binding of such a GalacDock to a GalacTag displayed on an NP surface thus provides an NP displaying the target moiety on the exterior NP surface, via the GalacTag/GalacDock binding pair.

Thus one aspect of the present invention is a method of producing NPs displaying a target moiety on the NP exterior surface, by (a) providing an NP displaying a GalacTag on the exterior surface, and then (b) contacting said NP with the corresponding GalacDock of the GalacTag/GalacDock binding pair, where the GalacDock is attached to the desired target moiety, and where said contact occurs under conditions that allow the formation of an isopeptide bond between the GalacDock and GalacTag. The target moiety may be selected from (a) antigenic non-polypeptide molecules (e.g., glycans or saccharides), (b) antigenic polypeptides, and (c) antigenic glycoconjugate molecules. Multiple copies of structurally defined antigenic epitopes can be displayed on the exterior surface of NPs using the GalacDock/GalacTag system of the present invention.

Recombinantly expressed bacteriophage Qbeta coat protein forms uniform nanoparticles (see, e.g., Brown et al., Biochemistry (2009) 48(47):11155-7); such Qbeta particles may be used in the present invention. Various bacterial polypeptides can also produce nanoparticles. Jardine et al. reported LS from the bacterium Aquifex aeolicus fused to an HIV gp120 antigen self-assembled into a 60-mer nanoparticle. Jardine et al., Science 340:711-716 (2013). Jardine et al. described use of mammalian cells to produce LS nanoparticles comprising the HIV gp120 antigen.

The i301 nanoparticle is based on 2-keto-3-deoxy-phosphogluconate (KDPG) aldolase from the hyperthermophilic bacterium Thermotoga maritima, and is contemplated for use in the present invention. The i301 has amino acid mutations that alter the interface between the wild-type protein trimer and promote assembly into a higher order dodecahedral 60-mer (Hsia et al., Design of a Hyperstable 60-Subunit Protein Icosahedron. Nature 2016, 535:136-139). The mi3 nanoparticle is based on the i301 system, but with further mutations of two surface-exposed cysteines to avoid potential disulfide bond formation (see, e.g., Bruun et al., ACS Nano (2018) 12(9):8855-8866).

Ferritin is an iron transport and storage protein found in both prokaryotes and eukaryotes. Mammalian ferritin is typically composed of 24 polypeptide subunits of ferritin heavy (H) and light (L) chains that self-assemble into a roughly spherical structure. Bacterial ferritin typically has a single polypeptide subunit type. H. pylori bacterial ferritin consists of 24 identical polypeptide subunits that self-assemble into a spherical nanoparticle. Li et al. reported preparation of a nucleotide sequence encoding a fusion of bacterial (H. pylori) ferritin subunit polypeptide and a rotavirus VP6 antigen. The expressed fusion polypeptides are described as self-assembling into spherical NPs displaying the rotavirus capsid protein VP6, and capable of inducing an immune response in mice. (Li et al., J Nanobiotechnol 17:13 (2019)). Wang et al. designed chimeric polypeptides comprising H. pylori ferritin and antigenic peptides from N. gonorrhoeae; the chimeric polypeptide is described as assembling into a 24-mer nanoparticle displaying the antigenic peptides on the NP exterior surface. (Wang et al., FEBS Open Bio 7(8):1196 (2017)). Kanekiyo et al. described a self-assembling recombinant bacterial (H. pylori) ferritin nanoparticle (24-mer), comprising fusions of the ferritin subunit polypeptide and influenza HA antigenic peptides, which displayed influenza HA trimers on its surface. (Kanekiyo et al., Nature 499(7456):102 (2013)). Nanoparticles based on insect ferritin and comprising both heavy and light chain subunit polypeptides have been described for use in displaying, on the NP surface, trimeric antigens (WO2018/005558 (PCT/US2017/039595), Kwong et al.). Li et al. described a nanoparticle made of recombinant fusion polypeptides comprising a human ferritin light-chain subunit and a short HIV-1 antigenic peptide attached to the amino terminus of the ferritin light-chain sequence, with self-assembly of these fusion polypeptides placing the HIV-1 antigenic peptide at the exterior surface of the NP. Li et al., Ind. Biotechnol. 2:143-47 (2006)).

NPs may also be based on GBS (Streptococcus agalactiae) ferritin proteins, such as ferritin from GBS LMG Strain 14747 (isolated from a bovine source) or from GBS DK-PW-092 (isolated from a human source). Recombinantly produced ferritin proteins may be modified while retaining the ability to self-assemble into NPs. One such modification is the addition of a His-tag, such as a C-terminal His tag attached by a flexible linker, to aid in purification. Additional amino acid modifications may be made to aid in production, stability and/or yield, for example, to replace cysteine residues that do not establish disulfide bridges (and thus may cause aggregation), or to add N-terminal helical portions to promote the colloidal stability and yield of multimeric particles. A “GB S ferritin subunit polypeptide”, as used herein, refers to a GBS ferritin NP subunit polypeptide from any GBS strain unless otherwise denoted.

Thus NP polypeptide subunits useful in combination with the GalacDock/GalacTag system of the present invention may comprise or consist of a naturally-occurring GBS ferritin polypeptide from any GBS strain, or modifications or variants of such polypeptides that retain the ability to self-assemble into a NP. The sequence of the GB S subunit per se may be modified in comparison to a naturally-occurring GB S sequence, e.g., by amino acid deletions, insertions, or substitutions; to such modified subunit sequences may further be added one or more N-terminal or C-terminal sequences, such as a purification tag.

Molecules, including antigenic molecules, attached to the exterior surface of an NP may be referred to herein as “display” or “displayed” molecules. Antigen-displaying nanoparticles preferably display multiple copies of antigenic molecules in an ordered array. It is theorized that an ordered multiplicity of antigens presented on a NP allows multiple binding events to occur simultaneously between the NP and host cells, which favors the induction of a potent host immune response. See e.g., Lopez-Sagaseta et al., Compu and Structural Biotech J, 14:58-68 (2016).

### Polynucleotide Molecules.

A further aspect of the present invention is methods of producing the polypeptides of the present invention using recombinant DNA methods, including fusions of GalacDock or GalacTag polypeptides with polypeptide target moieties (e.g. by operably linking a nucleotide sequence encoding a GalacDock or GalacTag peptide with a nucleotide sequence encoding the polypeptide target moiety, and expressing the protein product). Accordingly, the present invention provides polynucleotide molecules encoding the polypeptides of the present invention, including polynucleotides coding for: GalacTags, fusions of a GalacTag with a polypeptide target moiety, GalacDocks, and fusions of a GalacDock with a polypeptide target moiety. One such fusion protein is an NP subunit polypeptide attached to a GalacTag sequence; such fusion proteins that self-assemble into NPs displaying GalacTags may themselves be referred to as NP subunit polypeptides. The polynucleotide molecule may comprise RNA or DNA. Such recombinant nucleic acid sequences may comprise additional sequences useful for promoting expression or purification of the encoded polypeptide.

Polynucleotide molecules encoding the polypeptides of the invention may be codon optimized for expression in a selected prokaryotic or eukaryotic host cell. By “codon optimized” is intended modification with respect to codon usage that may increase translation efficacy and/or half-life of the nucleic acid.

### Expression Methods

The polypeptides of the present invention can be produced any suitable means, including by recombinant expression production or by chemical synthesis. Polypeptides of the invention may be recombinantly expressed and purified using any suitable method as is known in the art, and the product analyzed using methods as known in the art, e.g., by crystallography, Dynamic Light Scattering (DLS), Nano-Differential Scanning Fluorimetry (Nano-DSF), and Electron Microscopy, to confirm modified sequences assemble into nanoparticles.

The present invention further provides a process for producing polypeptides of the invention, which comprises (a) transforming or transfecting a suitable host cell with a vector which comprises a nucleotide sequence encoding the desired polypeptide, and then (b) culturing the host cell under conditions which allow expression of the polypeptide. The method may further comprise recovering, isolating, or purifying the expressed polypeptide. The expressed polypeptide may be a GalacDock or GalacTag polypeptide, which may be produced attached or linked to a target moiety, such as another polypeptide. In one embodiment, where the expressed polypeptide comprises an NP subunit polypeptide, multiple copies of such subunit polypeptides may be expressed in a host cell where they self-assemble into a multimeric nanoparticle within the host cell. The assembled NP can then be recovered, isolated or purified from the cell or the culture medium in which the cell is grown.

Methods of recombinant expression suitable for the production of the polypeptides of the present invention are known in the art. The expressed polypeptide may include a purification tag, a linker, or a “target” polypeptide (as described herein). Various expression systems are known in the art, including those using human (e.g., HeLa) host cells, mammalian (e.g., Chinese Hamster Ovary (CHO)) host cells, prokaryotic host cells (e.g., E. coli), or insect host cells. The host cell is typically transformed with the recombinant nucleic acid sequence encoding the desired polypeptide product, cultured under conditions suitable for expression of the product, and the product purified from the cell or culture medium. Cell culture conditions are particular to the cell type and expression vector, as is known in the art.

When a recombinant host cell of the present invention is cultured under suitable conditions, the recombinant nucleic acid expresses a polypeptide as described herein. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster)), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof.

Host cells can be cultured in conventional nutrient media modified as appropriate and as will be apparent to those skilled in the art (e.g., for activating promoters). Culture conditions, such as temperature, pH and the like, may be determined using knowledge in the art, see e.g., Freshney (1994) Culture of Animal Cells, a Manual of Basic Technique, third edition, Wiley-Liss, New York and the references cited therein. In bacterial host cell systems, a number of expression vectors are available including, but not limited to, multifunctional E. coli cloning and expression vectors such as BLUESCRIPT (Stratagene) or pET vectors (Novagen, Madison Wis.). In mammalian host cell systems, a number of expression systems, including both plasmids and viral-based systems, are available commercially.

Eukaryotic or microbial host cells expressing polypeptides of the invention can be disrupted by any convenient method (including freeze-thaw cycling, sonication, mechanical disruption), and polypeptides and/or NPs can be recovered and purified from recombinant cell culture by any suitable method known in the art (including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography). High performance liquid chromatography (HPLC) can be employed in the final purification steps.

In general, and using methods as are known in the art, expression of a recombinantly encoded polypeptide of the present invention involves preparation of an expression vector comprising a recombinant polynucleotide under the control of one or more promoters, such that the promoter stimulates transcription of the polynucleotide and promotes expression of the encoded polypeptide. “Recombinant Expression” as used herein refers to such a method.

In a further aspect, the present invention provides recombinant expression vectors comprising a recombinant nucleic acid sequence of any embodiment of the invention operatively linked to a suitable control sequence, such as a promoter capable of directing expression of the coding sequence in a selected host cell. “Recombinant expression vector” includes vectors that operatively link a nucleic acid coding region or gene to any control sequences capable of effecting expression of the gene product. “Control sequences” are nucleic acid sequences capable of effecting the expression of the nucleic acid molecules and need not be contiguous with the nucleic acid sequences, so long as they function to direct the expression thereof. Recombinant expression vectors can be of any type known in the art, including but not limited to plasmid and viral-based expression vectors. The control sequence used to drive expression of the disclosed nucleic acid sequences in a mammalian system may be constitutive or inducible. The construction of expression vectors for use in transfecting prokaryotic cells is also well known in the art. (See, for example, Sambrook, Fritsch, and Maniatis, in: Molecular Cloning, A Laboratory Manual, Cold Spring Harbor Laboratory Press, 1989; Gene Transfer and Expression Protocols, pp. 109-128, ed. E. J. Murray, The Humana Press Inc., Clifton, N.J.), and the Ambion 1998 Catalog (Ambion, Austin, Tex.). The expression vector must be replicable in the selected host organism either as an episome or by integration into host chromosomal DNA. In non-limiting embodiments, the expression vector is a plasmid vector or a viral vector. Expression vectors suitable for use in a given host-expression system and containing the encoding nucleic acid sequence and transcriptional/translational control sequences, may be made by any suitable technique as is known in the art. Typical expression vectors contain suitable promoters, enhancers, and terminators that are useful for regulation of the expression of the coding sequence(s) in the expression construct. The vectors may also comprise selection markers to provide a phenotypic trait for selection of transformed host cells (such as conferring resistance to antibiotics such as ampicillin or neomycin). Nucleic acid or vector modification may be undertaken in a manner known by the art, see e.g., WO 2012/049317 (corresponding to US 2013/0216613) and WO 2016/092460 (corresponding to US 2018/0265551). For example, the nucleic acid sequence encoding a polypeptide as described herein is cloned into a vector suitable for introduction into the selected cell system, e.g., bacterial or mammalian cells (e.g., CHO cells). Transformed cells are expanded, e.g., by culturing.

In a further embodiment, the present invention provides recombinant host cells that comprise a recombinant expression vector of the present invention. Suitable host cells can be either prokaryotic or eukaryotic, such as mammalian cells. The cells can be transiently or stably transfected. Such transfection of expression vectors into prokaryotic and eukaryotic cells can be accomplished via any technique known in the art, including but not limited to standard bacterial transformations, calcium phosphateco-precipitation, electroporation, or liposome mediated-, DEAE dextran mediated-, polycationic mediated-, or viral mediated transfection or transduction. (See, for example, Molecular Cloning: A Laboratory Manual (Sambrook, et al., 1989, Cold Spring Harbor Laboratory Press; Culture of Animal Cells: A Manual of Basic Technique, 2.sup.nd Ed. (R. I. Freshney. 1987. Liss, Inc. New York, N.Y.).

### Purification

The term “purified” as used herein refers to the separation or isolation of a defined product (e.g., a recombinantly expressed polypeptide) from a composition containing other components (e.g., a host cell or host cell medium). A polypeptide composition that has been fractionated to remove undesired components, and which composition retains its biological activity, is considered purified. A purified polypeptide retains its biological activity. Purified is a relative term and does not require that the desired product be separated from all traces of other components. Stated another way, “purification” or “purifying” refers to the process of removing undesired components from a composition or host cell or culture. Various methods for use in purifying polypeptides and NPs of the present invention are known in the art, e.g., centrifugation, dialysis, chromatography, gel electrophoresis, affinity purification, filtration, precipitation, antibody capture, and combinations thereof. The polypeptides of the present invention may be expressed with a tag operable for affinity purification, such as a 6xHistidine tag as is known in the art. A His-tagged polypeptide may be purified using, for example, Ni-NTA column chromatography or using anti-6xHis antibody fused to a solid support.

Thus, the term “purified” does not require absolute purity; rather, it is intended as a relative term. A “substantially pure” preparation of polypeptides (or nanoparticles) or nucleic acid molecules is one in which the desired component represents at least 50% of the total polypeptide (or nucleic acid) content of the preparation. In certain embodiments, a substantially pure preparation will contain at least 60%, at least 70%, at least 80%, at least 85%, at least 90%, at least 95%, at least 97%, at least 98%, or at least 99% or more of the total polypeptide (or nucleic acid) content of the preparation. Methods for quantifying the degree of purification of expressed polypeptides are known in the art and include, for example, determining the specific activity of an active fraction, or assessing the number of polypeptides within a fraction by SDS/PAGE analysis.

Thus, in the sense of the present invention, a “purified” or an “isolated” biological component (such as a polypeptide, an NP, or a nucleic acid molecule) has been substantially separated or purified away from other biological components in which the component naturally occurs or was recombinantly produced. The term embraces polypeptides, NPs, and nucleic acid molecules prepared by chemical synthesis as well as by recombinant expression in a host cell.

The polypeptides of the present invention may contain an amino acid sequence known as a “tag” (distinct from a GalacTag), which facilitates purification (e.g. a polyhistidine-tag to allow purification on a nickel-chelating resin). Examples of affinity-purification tags include, e.g., 6xHis tag (hexahistidine, binds to metal ion)(SEQ ID NO: 34), maltose-binding protein (MBP) (binds to amylose), glutathione-S-transferase (GST) (binds to glutathione), FLAG tag (Asp-Tyr-Lys-Asp-Asp-Asp-Asp-Lys (SEQ ID NO: 35), binds to an anti-flag antibody), Strep tag (Ala-Trp-Arg-His-Pro-Gln-Phe-Gly-Gly (SEQ ID NO: 36), or Trp-Ser-His-Pro-Gln-Phe-Glu-Lys (SEQ ID NO: 37), or Trp-Ser-His-Pro-Gln-Phe-Glu-Lys-Gly-Gly-Gly-Ser-Gly-Gly-Gly-Ser-Gly-Gly-Gly-Ser-Trp-Ser-His-Pro-Gln-Phe-Glu-Lys binds to streptavidin (SEQ ID NO: 38). The tag may be removed (enzymatically or through other means) prior to use of the polypeptide, or may be retained.

### Methods of Conjugation

GalacDock and GalacTag polypeptides may be attached to target molecules by any suitable means.

Genetic construct: In one embodiment, a nucleotide construct is prepared that recombinantly expresses a contiguous polypeptide sequence comprising both the GalacDock (or GalacTag) sequence and the polypeptide sequence of the target molecule, i.e., expresses a fusion polypeptide comprising both the Galac polypeptide and the target polypeptide. Where the target peptide is an NP subunit polypeptide, the encoded fusion polypeptides are capable of self-assembly into an NP.

Chemical conjugation: Functional groups present on the polypeptides of the invention can be used for site-specific conjugation of target molecules. Amino acid side-chain groups used for conjugation include amino group on lysine, thiol on cysteine, carboxylic acid on aspartic acids and glutamic acids, and hydroxyl moiety on tyrosine. Heterobifunctional crosslinkers are available for protein conjugation. Primary amines on proteins can be conjugated to carboxylic acids on another protein using 1-ethyl-3-(-3-dimethylaminopropyl) carbodiimide (EDC) crosslinkers, typically in combination with N-hydroxysuccinimide (NHS). One or more selected amino acid residues within a polypeptide sequence may be modified using methods known in the art to provide a site suitable for chemical conjugation, where such modification does not disrupt the polypeptide activity.

### Antigens

In one embodiment of the present invention the GalacTag or GalacDock moiety is joined to a target molecule that is an antigenic molecule. The antigenic molecule may be a poly- or oligo-saccharide, such as a bacterial capsular polysaccharide; the saccharide may be linked to a carrier protein to provide a glycoconjugate. As used herein, the term ‘carrier protein’ in reference to a glycoconjugate does not include GalacDock or GalacTag polypeptides of the invention. Carrier proteins may enhance the immunogenicity of an oligo- or polysaccharide antigen. Diphtheria toxoid (DT), tetanus toxoid (TT) and CRM197 (detoxified DT variant) are in use in commercial vaccines as carrier proteins for polysaccharide antigens. (CRM stands for Cross Reacting Material). Thus the carrier protein may be selected from tetanus toxoid (TT), diphtheria toxoid (DT), or derivatives thereof such as CRM197 or other detoxified variants of DT. Other suitable carrier proteins include EPA (exotoxin A of pseudomonas), the N. meningitidis outer membrane protein (see e.g., EP-A-0372501), synthetic peptides (see e.g., EP-A-0378881, EP-A 0427347), heat shock proteins (see e.g., WO93/17712, WO94/03208), pertussis proteins (see e.g., WO98/58668, EP A 0471177), cytokines (see e.g., WO91/01146), lymphokines (see e.g., WO91/01146), hormones (see e.g., WO91/01146), growth factors (see e.g., WO91/01146), artificial proteins comprising multiple human CD4+ T cell epitopes from various pathogen-derived antigens, protein D from H. influenzae (see e.g., EP-A-0594610, WO00/56360), pneumolysin, pneumococcal surface protein PspA (see e.g., WO02/091998), iron-uptake proteins, toxin A or B from C. difficile (see e.g., WO00/61761).

In some embodiments of the present invention, the antigenic molecule is an oligo/polysaccharide derived from a bacterial pathogen and in particular may be derived from bacterial capsular saccharide or lipooligosaccharide (LOS) or lipopolysaccharide (LPS). For example, the oligo/polysaccharide may be derived from a bacterial pathogen selected from the group consisting of: S. agalactiae, Haemophilus influenzae type b (“Hib”); Neisseria meningitidis (including serotypes A, C, W and/or Y); Streptococcus pneumoniae (including serotypes 1, 2, 3, 4, 5, 6A, 6B, 7F, 8, 9N, 9V, 10A, 11A, 12F, 14, 15B, 15C, 17F, 18C, 19A, 19F, 20, 22F, 23F and/or 33F); Staphylococcus aureus, Bordetella pertussis, and Salmonella species, Pseudomonas aeruginosa, Enterococcus faecalis or E. faecium (trisaccharide repeats), Yersinia species, Vibrio cholerae, Klebsiella species. Another saccharide which may be included is the Streptococcus pyogenes group-specific antigen (GAS carbohydrate).

Further bacterial antigens for use in the present invention include those from by Escherichia species, Shigella species, Helicobacter species, Proteus species, Pseudomonas species, Corynebacterium species, Streptomyces species, Streptococcus species, Enterococcus species, Staphylococcus species, Bacillus species, Clostridium species, Listeria species, or Campylobacter species.

### Group B Streptococcus Antigens

In one embodiment of the present invention, the antigenic molecule attached to a GalacTag is a GBS capsular polysaccharide or immunogenic fragment thereof. The GBS capsular polysaccharide may be selected from any serotype, including Ia, Ib, II, III, IV and V.

GBS is a β-hemolytic, encapsulated Gram-positive microorganism that is a major cause of neonatal sepsis and meningitis. The GBS capsule is a virulence factor that assists the bacterium in evading human innate immune defenses. The GBS capsule consists of high molecular weight polymers made of multiple identical repeating units of four to seven monosaccharides and including sialic acid (N-acetylneuraminic acid) residues. GBS can be classified into ten serotypes (Ia, Ib, II, III, IV, V, VI, VII, VIII, and IX) based on the chemical composition and the pattern of glycosidic linkages of the capsular polysaccharide repeating units. The capsular polysaccharides of different serotypes are chemically related, but are antigenically different. Non-typeable strains of GBS are also known to exist. Description of the structure of GBS CPS may be found in the published literature (see e.g., WO2012/035519). GBS capsular polysaccharides used as antigens may be chemically modified or depolymerized (see e.g., WO2006/050341).

The term “saccharide” as used herein refers to polysaccharides (PS) and oligosaccharides. A GBS serotype polysaccharide, as used herein, refers to the GBS bacterial capsular polysaccharide of that serotype. Isolated GBS serotype polysaccharides may be “sized,” i.e., their molecular weight reduced compared to the starting wild-type polysaccharide, by known methods (see for example EP497524 and EP497525). Methods of sizing polysaccharides include acid hydrolysis treatment, hydrogen peroxide treatment, sizing by EMULSIFLEX followed by a hydrogen peroxide treatment to generate saccharide fragments, and microfluidization. The weight-average molecular weight (Mw) of the saccharide is as measured by MALLS (Multi-Angle Laser Light Scattering).

The GBS saccharide antigenic molecule attached to a GalacTag may be chemically modified relative to the capsular saccharide as found in nature. For example, the saccharide may be de-O-acetylated (partially or fully), de-N-acetylated (partially or fully), N-propionylated (partially or fully), etc. De-acetylation may occur before, during or after conjugation, but preferably occurs before conjugation. Depending on the particular saccharide, chemical modification such as de-acetylation may or may not affect immunogenicity; the effect of chemical modification on antigen immunogenicity can be assessed by routine assays. When the saccharide antigen is the Streptococcus agalactiae serotype V capsular saccharide, then the saccharide antigen may be modified as described in WO2006/050341. In particular, the Streptococcus agalactiae serotype V capsular saccharide may be desialylated.

The GBS saccharide antigenic molecule attached to a GalacTag may be an oligosaccharide fragment of the native GBS polysaccharide, such as a depolymerized fragment of the native GBS polysaccharide. The antigenicity of such fragments can be assessed by routine assays.

The GBS antigenic molecule attached to a GalacTag may be a chimeric GBS capsular polysaccharide (see, e.g., WO2017/001586). Such chimeric capsular polysaccharides may comprise at least one capsular polysaccharide repeating unit of a first serotype and at least one capsular polysaccharide repeating unit of a second, different serotype wherein the repeating units are joined by a glycosidic bond.

Nearly all GBS strains express a protein which belongs to the so-called alpha-like proteins (Alps), of which Cα, Alp1, Alp2, Alp3, Rib, and Alp4 are known to occur in GBS. See e.g. Maeland et al., Clin Vaccine Immunol 22(2): 153-59 (2015). GBS protein antigens, including Alp3, and Rib proteins have been investigated as vaccine components (see e.g., Gravekamp et al., Infect Immun 67:2491-6 (1999); Erdogan et al., Infect Immun 70:803-11 (2002).) Additional GBS protein antigens include immunogenic fusion proteins comprising the N-terminal regions of two or more GBS proteins (such as Rib, AlpC, Alp1, Alp2, Alp3 or Alp4) (see e.g., WO2017/068112; WO2008/127179).

In one embodiment of the present invention, the antigenic molecule attached to a GalacTag or GalacDock polypeptide is a heterologous antigenic GBS protein, such as a GBS surface protein.

### Compositions

A further embodiment of the present invention is pharmaceutical compositions and immunogenic compositions, such as vaccines. The compositions are suitable for administration to a human or non-human mammalian subject, and comprise (a) a bound GalacTag/GalacDock pair of the present invention, where at least one of the GalacTag or GalacDock polypeptides is attached to a target molecule having an immunogenic or therapeutic effect, and (b) a pharmaceutically acceptable diluent, carrier, or excipient. An “immunogenic composition” is a composition of matter suitable for administration to a human or non-human mammalian subject and which, upon administration of an immunologically effective amount, elicits a specific immune response, e.g., against an antigen bound to a GalacTag polypeptide, a GalacDock polypeptide, or a GalacTag/GalacDock pair. As such, an immunogenic composition includes one or more antigens (for example, polypeptide antigens or saccharide antigens from Group B Streptococcus), or immunogenic fragments or antigenic epitopes thereof. An immunogenic composition can also include one or more additional components, such as an adjuvant capable of enhancing an immune response, or an additional excipient or carrier.

In certain instances, immunogenic compositions are administered to elicit an immune response that protects the subject against infection by a pathogen, or decreases symptoms or conditions induced by a pathogen.

Numerous pharmaceutically acceptable diluents and carriers and/or pharmaceutically acceptable excipients are known in the art and are described, e.g., in Remington's Pharmaceutical Sciences, by E. W. Martin, Mack Publishing Co., Easton, Pa., 15th Edition (1975). The adjective “pharmaceutically acceptable” indicates that the diluent, or carrier, or excipient, is suitable for administration to a subject (e.g., a human or non-human mammalian subject). In general, the nature of the diluent, carrier and/or excipient will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. In certain formulations (for example, solid compositions, such as powder forms), a liquid diluent is not employed. In such formulations, non-toxic solid or gel carriers can be used, including for example, pharmaceutical grades of trehalose, mannitol, lactose, starch or magnesium stearate. Suitable carriers are typically large, slowly metabolized macromolecules such as proteins, polysaccharides, polylactic acids, polyglycolic acids, polymeric amino acids, amino acid copolymers, lipid aggregates (such as oil droplets or liposomes), and inactive virus particles. Such carriers are known in the art.

The immunogenic compositions of the invention may be administered by conventional routes, such as parenterally, e.g., by injection, either subcutaneously, intraperitoneally, transdermally, or intramuscularly.

Injectable or implantable depot formulations that provide sustained release of the immunogenic construct may be utilized in compositions of the present invention. Such formulations comprise a matrix material and the immunogenic construct. The matrix may be a gel, colloidal dispersion, hydrogel, or a composition that becomes a gel, colloidal dispersion, or hydrogel when placed in contact with living tissue and the fluids therein. The immunogenic construct may be encapsulated in microparticles, such as silica microparticles, which may be embedded in a silica gel. See e.g., Shoichet et al., Journal of Controlled Release, 160: 666-675 (2012); Shoichet et al., Journal of Controlled Release, 166:197-202 (2013); US2009/0324695; Wang et al., Biomaterials, 31:4955-4951 (2010); US 2016/0136088.

Accordingly, suitable excipients and carriers can be selected by those of skill in the art to produce a formulation suitable for delivery to a subject by a selected route of administration.

Dosage treatment may be a single dose schedule or a multiple dose schedule. The immunogenic composition may be administered in conjunction with other immunoregulatory agents. Any suitable route of administration can be used and administered according to any suitable schedule.

Immunogenic compositions of the present invention may additionally include one or more adjuvants. An “adjuvant” is an agent that enhances the production of an immune response in a non-specific manner Common adjuvants include suspensions of minerals (alum, aluminum hydroxide, aluminum phosphate); saponins such as QS21; emulsions, including water-in-oil, and oil-in-water (and variants thereof, including double emulsions and reversible emulsions), liposaccharides, lipopolysaccharides, immunostimulatory nucleic acid molecules (such as CpG oligonucleotides), liposomes, Toll Receptor agonists (particularly, TLR2, TLR4, TLR7/8 and TLR9 agonists), and various combinations of such components.

Preparation of immunogenic compositions, such as vaccines, including those for administration to human subjects, is generally described in Pharmaceutical Biotechnology, Vol. 61 Vaccine Design—the subunit and adjuvant approach, edited by Powell and Newman, Plenum Press, 1995. New Trends and Developments in Vaccines, edited by Voller et al., University Park Press, Baltimore, Md., U.S.A. 1978.

### Prophylactic and Therapeutic Uses

A further aspect of the present invention is a method of inducing an immune response in a subject, where said immune response is specific for an antigenic target molecule attached to a GalacDock, a GalacTag, or a GalacDock/GalacTag pair of the present invention, by administering to a subject an amount sufficient to provide an immunologically effective amount of the antigenic target molecule. The isopeptide bound GalacDock/GalacTag pair may comprise a nanoparticle. In one embodiment the antigenic molecule is a bacterial antigen; the subject may have a bacterial infection at the time of administration, or the administration may be given prophylactically to a subject who does not have a bacterial infection at the time of administration.

A further aspect of the present invention is a method of treating and/or preventing an infection, such as a bacterial infection, of a subject comprising administering to the subject an immunogenic composition as described herein, wherein the immune response provoked is a therapeutic or prophylactic immune response. In a specific embodiment, compositions described herein and comprising GBS antigens are used in the prevention of infection of a subject (e.g., a human subject) by Group B Streptococcus (S. agalacteriae).

Thus, in one embodiment, the compositions of the present invention are utilized in methods of immunizing a subject to achieve a protective (prophylactic) immune response, or as a therapeutic measure (i.e., directed against an existing disease in the subject).

### EMBODIMENTS

The various features which are referred to in individual sections above apply, as appropriate, to other sections. Consequently, features specified in one section may be combined with features specified in other sections, as appropriate. Those skilled in the art will recognize or be able to ascertain using no more than routine experimentation, many equivalents to the specific embodiments of the invention (or aspects of the disclosure) described herein. Such equivalents are intended to be encompassed by the following:

1. A polypeptide comprising or consisting of an amino acid sequence selected from: SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, and SEQ ID NO: 51.

2. The polypeptide of 1, comprising or consisting of amino acids 1-419 of SEQ ID NO:28.

3. The polypeptide of 1, comprising or consisting of a sequence having at least 90%, at least 92%, at least 95%, at least 97%, at least 98%, or at least 99% identity to amino acids 1-419 of SEQ ID NO:28.

4. A fusion protein comprising a polypeptide of 1 and a heterologous polypeptide.

5. The fusion protein of 4, where said heterologous polypeptide is covalently linked to the N-terminal amino acid of the polypeptide of 1, either directly or via an amino acid linker.

6. The fusion protein of 4, where said heterologous polypeptide is covalently linked to the C-terminal amino acid of the polypeptide of 1, either directly or via an amino acid linker.

7. The fusion protein of 4 where said heterologous polypeptide is an antigenic polypeptide.

8. The fusion protein of 4 where said heterologous antigenic polypeptide is an antigenic GBS surface protein or immunogenic fragment thereof.

9. A polypeptide of 1 conjugated to a bacterial capsular polysaccharide.

10. The conjugate of 9, where said bacterial capsular polysaccharide is also conjugated to a carrier protein.

11. The polypeptide of 1, conjugated to a detectable label.

12. A polypeptide comprising or consisting of an amino acid sequence selected from: SEQ ID NO: 8, SEQ ID NO:9, SEQ ID NO: 10, SEQ ID NO: 11, SEQ ID NO: 12, SEQ ID NO: 13, SEQ ID NO: 15, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, and SEQ ID NO: 60.

13. The polypeptide of 12, comprising or consisting of amino acids 422-452 of SEQ ID NO: 28.

14. The polypeptide of 12, comprising or consisting of a sequence having at least 90%, at least 93%, or at least 96% identity to amino acids 422-452 of SEQ ID NO: 28.

15. A fusion protein comprising a polypeptide of 12 and a heterologous polypeptide.

16. The fusion protein of 15, where said heterologous polypeptide is covalently linked to the C-terminal amino acid of the polypeptide of 10, either directly or via an amino acid linker.

17. The fusion protein of 15, where said heterologous polypeptide is covalently linked to the N-terminal amino acid of the polypeptide of 10, either directly or via an amino acid linker.

18. The fusion protein of 15 where said heterologous polypeptide is an antigenic polypeptide.

19. The fusion protein of 18 where said heterologous antigenic polypeptide is an antigenic GBS surface protein or immunogenic fragment thereof.

20. The polypeptide of 12 conjugated to a bacterial capsular polysaccharide.

21. The conjugate of 20, where said bacterial capsular saccharide is also conjugated to a carrier protein.

22. The polypeptide of 12, conjugated to a detectable label.

23. The fusion protein of 15 where said heterologous polypeptide is a polypeptide subunit of a self-assembling protein nanoparticle.

24. The fusion protein of 23 where said polypeptide subunit of a self-assembling protein nanoparticle is a GBS ferritin polypeptide subunit.

25. The fusion protein of 24 where said polypeptide subunit is selected from a sequence comprising or consisting of SEQ ID NO: 30, SEQ ID NO: 31, SEQ ID NO: 32, and SEQ ID NO: 33.

26. A peptide tag and binding partner pair wherein
(a) said peptide tag is a fragment of an isopeptide protein, said tag having a length of at least 5 amino acids but no more than 50 amino acids, and comprising a first reactive residue involved in formation of an intramolecular isopeptide bond in said isopeptide protein, wherein said peptide tag is either unconjugated or is conjugated to a heterologous polypeptide or to another molecule, and wherein said isopeptide protein is a Group B Streptococcus (GBS) pilus protein selected from the group consisting of (i) a Group B Streptococcus (GBS) pilus Backbone Protein (BP) or a protein with at least 95% identity thereto; and (ii) a Group B Streptococcus (GBS) pilus Ancillary Protein (AP) or a protein with at least 95% identity thereto; and wherein said isopeptide protein is capable of spontaneously forming an isopeptide bond;
(b) said binding partner comprises a different fragment of said isopeptide protein, wherein said fragment is at least 20 amino acids in length and comprises a second reactive residue involved in said isopeptide bond in said isopeptide protein, wherein the binding partner does not include the first reactive residue of the peptide tag; and
(c) said peptide tag and binding partner, when contacted with each other under suitable conditions, bind to each other and form an isopeptide bond between the first and second reactive residues.

27. The peptide tag and binding partner pair of 26 where said Group B Streptococcus (GBS) pilus protein is selected from the group consisting of (i) Group B Streptococcus (GBS) pilus Backbone Protein BP-1, (ii) Group B Streptococcus (GBS) pilus Backbone Protein BP-2a, and (iii) Group B Streptococcus (GBS) pilus Backbone Protein BP-2b, Group B Streptococcus (GB S) pilus Ancillary Protein AP1-1, and (iv) Group B Streptococcus (GBS) pilus Ancillary Protein AP1-2a.

28. The peptide tag and binding partner pair of 26 where said Group B Streptococcus (GBS) pilus protein comprises a sequence selected from SEQ ID NO: 26, SEQ ID NO: 27, SEQ ID NO: 28, SEQ ID NO: 40, SEQ ID NO: 41, SEQ ID NO: 42, SEQ ID NO: 61 and SEQ ID NO: 62.

29. The peptide tag and binding partner pair of 26 wherein the tag comprises or consists of amino acids 422-452 of SEQ ID NO: 28 and the binding partner comprises or consists of amino acids 1-419 of SEQ ID NO:28.

30. The peptide tag and binding partner pair of 26 wherein the tag comprises or consists of a sequence having at least 90%, at least 93%, or at least 96% identity to amino acids 422-452 of SEQ ID NO: 28, and the binding partner comprises or consists of a sequence having at least 90%, at least 92%, at least 95%, at least 97%, at least 98%, or at least 99% identity to amino acids 1-419 of SEQ ID NO:28, where said peptide tag and binding partner pair spontaneously bind to each other and form an isopeptide bond.

31. The peptide tag and binding partner pair of 26 where one of said peptide tag or binding partner pair is conjugated to a solid support.

32. A protein nanoparticle comprising a polypeptide subunit according to any one of 23-25.

33. A protein nanoparticle according to 32, where said nanoparticle is covalently joined to a polypeptide according to any one of 1-11.

34. A kit comprising a peptide tag and binding partner pair of 26, where said peptide tag and binding partner are not covalently joined.

35. The kit of 34, where at least one of the peptide tag and binding partner pair are conjugated to a detectable label.

36. The kit of 34, where at least one of the peptide tag and binding partner pair are conjugated to a heterologous polypeptide.

37. The kit of 34, where at least one of the peptide tag and binding partner pair are conjugated to a solid support.

38. A kit comprising:
(a) a protein nanoparticle, where said protein nanoparticle comprises multiple polypeptide subunits, and at least one polypeptide subunit is covalently bound to a peptide tag according to 12; and
(b) the binding partner of said peptide tag;
where said peptide tag and binding partner are not covalently joined.

39. The kit of 38, where said protein nanoparticle is a GBS ferritin nanoparticle.

40. The kit of 38, where said polypeptide subunit is selected from a sequence comprising or consisting of SEQ ID NO: 30, SEQ ID NO: 31, SEQ ID NO: 32, and SEQ ID NO: 33.

41. A nucleic acid molecule encoding a polypeptide or fusion protein of any one of 1-25.

42. A vector comprising a nucleic acid molecule of 41.

43. A host cell comprising a nucleic acid molecule of 41 or a vector of 42.

44. A method of recombinantly producing a polypeptide, comprising expressing a nucleic acid molecule according to 41.

45. The method of 44, further comprising isolating or purifying the expressed polypeptide.

46. A method of producing a protein nanoparticle (NP) displaying a peptide tag on the NP exterior surface, where said peptide tag is a fragment of an isopeptide protein, said tag having a length of at least 5 amino acids but no more than 50 amino acids, and comprising a first reactive residue involved in formation of an intramolecular isopeptide bond in said isopeptide protein, and wherein said isopeptide protein is a Group B Streptococcus (GBS) pilus protein selected from the group consisting of (i) a Group B Streptococcus (GBS) pilus Backbone Protein (BP) or a protein with at least 95% identity thereto; and (ii) a Group B Streptococcus (GBS) pilus Ancillary Protein (AP) or a protein with at least 95% identity thereto; and wherein said isopeptide protein is and capable of spontaneously forming an isopeptide bond, said method comprising:
(a) recombinantly expressing fusion proteins of said peptide tag and a NP polypeptide subunit, in a host cell under conditions that allow self-assembly of said nanoparticle subunits into a NP; and
(b) isolating or purifying the NP.

47. The method according to 46 wherein said NP polypeptide subunit is a GBS ferritin polypeptide subunit.

48. The method according to 47, where said GBS ferritin polypeptide subunit comprises or consists of a sequence selected from SEQ ID NO: 30, SEQ ID NO: 31, SEQ ID NO: 32, and SEQ ID NO: 33.

49. A method of producing a fusion protein, comprising:
(a) providing a peptide tag that is a fragment of an isopeptide protein, said tag having a length of at least 5 amino acids but no more than 50 amino acids, and comprising a first reactive residue involved in formation of an intramolecular isopeptide bond in said isopeptide protein, wherein said peptide tag is either unconjugated or is conjugated to a heterologous polypeptide or to another molecule, and wherein said isopeptide protein is a Group B Streptococcus (GBS) pilus Backbone Protein (BP) or Ancillary Protein (AP);
(b) providing a peptide binding partner to said peptide tag, where said binding partner comprises a different fragment of said isopeptide protein, wherein said fragment is at least 20 amino acids in length and comprises a second reactive residue involved in said isopeptide bond in said isopeptide protein, wherein the binding partner does not include the first reactive residue of the peptide tag; and
(c) contacting said peptide tag and binding partner under conditions that allow the peptide tag and binding partner to form an isopeptide bond between the first and second reactive residues,
wherein at least one of said peptide tag and binding partner is covalently attached to a heterologous molecule.

50. The method according to 49 wherein said Group B Streptococcus (GBS) pilus protein is selected from the group consisting of (i) Group B Streptococcus (GBS) pilus Backbone Protein BP-1, (ii) Group B Streptococcus (GBS) pilus Backbone Protein BP-2a, and (iii) Group B Streptococcus (GBS) pilus Backbone Protein BP-2b, Group B Streptococcus (GB S) pilus Ancillary Protein AP1-1, and (iv) Group B Streptococcus (GBS) pilus Ancillary Protein AP1-2a.

51. The method according to 49 wherein said Group B Streptococcus (GBS) pilus protein comprises a sequence selected from SEQ ID NO: 26, SEQ ID NO: 27, SEQ ID NO: 28, SEQ ID NO: 40, SEQ ID NO: 41, SEQ ID NO: 42, SEQ ID NO: 61 and SEQ ID NO: 62.

52. The method according to 49 wherein said peptide tag comprises or consists of amino acids 422-452 of SEQ ID NO: 28 and said peptide binding partner comprises or consists of amino acids 1-419 of SEQ ID NO:28.

53. The method according to 49 wherein said peptide tag comprises or consists of a sequence having at least 90%, at least 93%, or at least 96% identity to amino acids 422-452 of SEQ ID NO: 28, and said peptide binding partner comprises or consists of a sequence having at least 90%, at least 92%, at least 95%, at least 97%, at least 98%, or at least 99% identity to amino acids 1-419 of SEQ ID NO:28, where said peptide tag and binding partner pair spontaneously bind to each other and form an isopeptide bond.

54. The method according to 49, where said heterologous molecule is an antigenic polypeptide.

55. The method according to 54, where said heterologous antigenic polypeptide is an antigenic GBS surface protein or immunogenic fragment thereof.

56. The method according to 49, where said heterologous molecule is a bacterial capsular polysaccharide.

57. The method according to 56, where said bacterial capsular polysaccharide is a GBS capsular polysaccharide.

58. The method according to 56, where said bacterial capsular polysaccharide is also conjugated to a carrier protein.

59. A method of producing a protein nanoparticle displaying target molecules on its exterior surface, comprising:
(a) providing a multiplicity of fusion proteins comprising peptide tags conjugated at the C-terminus to a NP polypeptide subunit, where said peptide tag is a fragment of an isopeptide protein, said tag having a length of at least 5 amino acids but no more than 50 amino acids, and comprising a first reactive residue involved in formation of an intramolecular isopeptide bond in said isopeptide protein, and wherein said isopeptide protein is a Group B Streptococcus (GB S) pilus Backbone Protein (BP) or Ancillary Protein (AP);
(b) allowing self-assembly of said fusion proteins to provide an NP displaying said peptide tags on the external surface of the NP;
(c) providing a multiplicity of peptide binding partners to said peptide tag, where said binding partner comprises a different fragment of said isopeptide protein, wherein said fragment is at least 20 amino acids in length and comprises a second reactive residue involved in said isopeptide bond in said isopeptide protein, wherein the binding partner does not include the first reactive residue of the peptide tag, and wherein each of said peptide binding partners is conjugated to a heterologous molecule; and
(c) contacting said NP and said peptide binding partners under conditions that allow the peptide tags displayed on the external surface of the NP and binding partners to form isopeptide bonds.

60. The method according to 59 wherein said Group B Streptococcus (GBS) pilus protein is selected from the group consisting of (i) Group B Streptococcus (GBS) pilus Backbone Protein BP-1, (ii) Group B Streptococcus (GBS) pilus Backbone Protein is BP-2a, and (iii) Group B Streptococcus (GBS) pilus Backbone Protein BP-2b, Group B Streptococcus (GBS) pilus Ancillary Protein AP1-1, and (iv) Group B Streptococcus (GBS) pilus Ancillary Protein AP1-2a.

61. The method according to 59 wherein said Group B Streptococcus (GBS) pilus protein comprises a sequence selected from SEQ ID NO: 26, SEQ ID NO: 27, SEQ ID NO: 28, SEQ ID NO: 40, SEQ ID NO: 41, SEQ ID NO: 42, SEQ ID NO: 61 and SEQ ID NO: 62.

62. The method according to 59 wherein said peptide tag comprises or consists of amino acids 422-452 of SEQ ID NO: 28 and said peptide binding partner comprises or consists of amino acids 1-419 of SEQ ID NO:28.

63. The method according to 59 wherein said peptide tag comprises or consists of a sequence having at least 90%, at least 93%, or at least 96% identity to amino acids 422-452 of SEQ ID NO: 28, and said peptide binding partner comprises or consists of a sequence having at least 90%, at least 92%, at least 95%, at least 97%, at least 98%, or at least 99% identity to amino acids 1-419 of SEQ ID NO:28, where said peptide tag and binding partner pair spontaneously bind to each other and form an isopeptide bond.

64. The method according to 59, where said heterologous molecule is an antigenic polypeptide.

65. The method according to 64, where said heterologous antigenic polypeptide is an antigenic GBS surface protein or immunogenic fragment thereof.

66. The method according to 59, where said heterologous molecule is a bacterial capsular polysaccharide.

67. The method according to 66, where said bacterial capsular polysaccharide is a GBS capsular polysaccharide.

68. The method according to 66, where said bacterial capsular polysaccharide is also conjugated to a carrier protein.

69. A pharmaceutical composition comprising a fusion protein according to any one of 15-19.

70. A pharmaceutical composition comprising a nanoparticle according to any one of 32-33.

71. Use of a polypeptide according to any one of 1-14, 20-22; a fusion protein according to any one of 15-19, 23-25; a binding pair according to any one of 26-31; a nanoparticle according to any one of 32-33; or a pharmaceutical composition according to any one of 69-70; for the manufacture of a medicament for inducing an immune response.

72. Use of a nanoparticle according to any one of 32-33, or a pharmaceutical composition of any one of 69-70, for inducing an immune response in a subject.

73. Use of a nanoparticle according to any one of 32-33, or a pharmaceutical composition of any one of 69-70, in the prevention or treatment of disease.

74. A method of inducing an immune response in a subject, comprising administering to the subject an immunologically effective amount of a polypeptide according to any one of 1-14, 20-22; a fusion protein according to any one of 15-19, 23-25; or a nanoparticle according to 32-33.

### Terms

To facilitate review of the various embodiments of this disclosure, the following explanations of terms are provided. Additional terms and explanations are provided in the context of this disclosure. Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

“Nanoparticles” as used herein refers to particles of less than about 100 nm in size (less than about 100 nm in maximum diameter for spherical, or roughly spherical, particles).

As used herein the terms “protein,” “peptide,” and “polypeptide” are used interchangeably. A protein, peptide, or polypeptide sequence refers to a contiguous sequence of two or more amino acids linked by a peptide bond. The proteins, peptides, and polypeptides of the invention may comprise L-amino acids, D-amino acids, or a combination thereof.

As used herein, “conjugation” references the coupling of one molecule to another, e.g., the joining of two polypeptides by covalent bond. “Conjugate” herein means two or more molecules (e.g., proteins, saccharides, glycoconjugates) which are attached to each other.

As used herein, a “NP polypeptide subunit”, or “NP subunit”, refers to a polypeptide that, in combination with other polypeptide subunits, self-assembles into a nanoparticle. The subunit may comprise a polypeptide sequence which extends from the surface of the assembled nanoparticle (i.e., is ‘displayed’ by the nanoparticle), a purification tag, or other modifications as are known in the art and that do not interfere with the ability to self-assemble into a nanoparticle.

As used herein, a “variant” polypeptide refers to a polypeptide having an amino acid sequence which is similar, but not identical to, a reference sequence, wherein the biological activity of the variant protein is not significantly altered. Such variations in sequence can be naturally occurring variations or they can be engineered through the use of genetic engineering techniques as known to those skilled in the art. Examples of such techniques may be found, e.g., in Sambrook et al., Molecular Cloning—A Laboratory Manual, 2nd Edition, Cold Spring Harbor Laboratory Press, 1989, pp. 9.31-9.57), or in Current Protocols in Molecular Biology, John Wiley & Sons, N.Y. (1989), 6.3.1-6.3.6.

As used herein, a “fusion polypeptide” or “chimeric polypeptide” is a polypeptide comprising amino acid sequences from at least two unrelated proteins that have been joined together, via a peptide bond, to make a single polypeptide. The unrelated amino acid sequences can be joined directly to each other or they can be joined using a linker sequence. As used herein, polypeptides are unrelated if their amino acid sequences are not normally found joined together via a peptide bond in their natural environment(s) (e.g., inside a cell). For example, a GB S pilin polypeptide is considered unrelated to a GB S ferritin subunit polypeptide.

As used herein, an “antigen” (or ‘antigenic’ molecule) is a molecule (such as a protein, saccharide, or glycoconjugate), a compound, composition, or substance that stimulates an immune response by producing antibodies and/or a T cell response in a mammal, including compositions that are injected, absorbed or otherwise introduced into a mammal. The term “antigen” includes all related antigenic epitopes. The term “epitope” or “antigenic determinant” refers to a site on an antigen to which B and/or T cells respond. The “predominant antigenic epitopes” are those epitopes to which a functionally significant host immune response, e.g., an antibody response or a T-cell response, is made. Thus, with respect to a protective immune response against a pathogen, the predominant antigenic epitopes are those antigenic moieties that when recognized by the host immune system result in protection from disease caused by the pathogen. The term “T-cell epitope” refers to an epitope that when bound to an appropriate MHC molecule is specifically bound by a T cell (via a T cell receptor). A “B-cell epitope” is an epitope that is specifically bound by an antibody (or B cell receptor molecule).

As used herein, the term “immunogenic” refers to the ability of a specific antigen, or a specific region thereof, to elicit an immune response to that antigen or region thereof when administered to a mammalian subject. The immune response may be humoral (mediated by antibodies) or cellular (mediated by cells of the immune system), or a combination thereof.

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”), such as a GBS antigen. A “protective immune response” is an immune response that inhibits a detrimental function or activity of a pathogen, prevents infection by a pathogen in an individual, or decreases symptoms that result from infection by the pathogen. A protective immune response can be measured, for example, by measuring resistance to pathogen challenge in vivo.

The term “fragment,” in reference to a polypeptide (or polysaccharide) antigen, refers to a contiguous portion (that is, a subsequence) of that polypeptide (or polysaccharide). An “immunogenic fragment” of a polypeptide or polysaccharide refers to a fragment that retains at least one immunogenic epitope (e.g., a predominant immunogenic epitope or a neutralizing epitope).

An “effective amount” means an amount sufficient to cause the referenced effect or outcome. An “effective amount” can be determined empirically and in a routine manner using known techniques in relation to the stated purpose. An “immunologically effective amount” is a quantity of an immunogenic composition sufficient to elicit an immune response in a subject (administered either in a single dose or in a series of doses). Commonly, the desired result is the production of an antigen (e.g., pathogen)-specific immune response that is capable of or contributes to protecting the subject against the pathogen. Obtaining a protective immune response against a pathogen can require multiple administrations of the immunogenic composition. Thus, in the context of this disclosure, the term immunologically effective amount encompasses a fractional dose that, in combination with previous or subsequent administrations, induces a protective immune response.

As used herein, a “glycoconjugate” is a carbohydrate moiety (such as a polysaccharide) covalently linked to a moiety that is a different chemical species, such as a protein, peptide, lipid or lipid. Conjugation of polysaccharide antigens to suitable protein carriers is known in the art to increase immunogenicity of the polysaccharide antigen; such glycoconjugates are known for use in vaccination.

As used herein, where a nucleic acid sequence is operably linked to another polynucleotide molecule that it is not associated with in nature, the two sequences are “heterologous” with regard to each other. Similarly, when a polypeptide is covalently linked to another polypeptide that it is not covalently linked to in nature, the two polypeptides are heterologous to each other. The covalent linkage of heterologous polypeptides may be direct, or may include a short linker or intervening sequence. Similarly, when a polypeptide is in a stable complex with another polypeptide that it is not found in a stable complex with in nature, the polypeptides are “heterologous” with regard to each other. A polypeptide (or nucleic acid) sequence that is “heterologous” to GBS refers to a polypeptide (or nucleic acid) sequence that is not found in naturally occurring GBS cells. Further, when a host cell comprises a nucleic acid molecule or polypeptide that it does not naturally comprise, the nucleic acid molecule and polypeptide may be referred to as “heterologous” to the host cell. For purposes of the present invention, in a fusion protein comprising two polypeptides from the same host organism (such as GBS), the polypeptides are considered heterologous to each other when they are not naturally covalently associated with each other. Thus, for example, a polypeptide comprising a GB S surface protein (or fragment thereof) attached to a GB S ferritin subunit polypeptide, producing a polypeptide not found in nature, would be considered a fusion protein of two heterologous polypeptide sequences.

“Operably linked” means connected so as to be operational, for example, in the configuration of recombinant polynucleotide sequences for protein expression. In certain embodiments, “operably linked” refers to the art-recognized positioning of nucleic acid components such that the intended function (e.g., expression) is achieved. A person with ordinary skill in the art will recognize that under certain circumstances, two or more components “operably linked” together are not necessarily adjacent to each other in the nucleic acid or amino acid sequence. A coding sequence that is “operably linked” to a control sequence (e.g., a promoter, enhancer, or IRES) is ligated in such a way that expression of the coding sequence is under the influence or control of the control sequence, but such a ligation is not limited to adjacent ligation.

By “adjacent”, it is meant “next to” or “side-by-side”. By “immediately adjacent”, it is meant adjacent to with no material structures in between (e.g., in the context of an amino acid sequence, two residues “immediately adjacent” to each other means there are atoms between the two residues sufficient to form the bonds necessary for a polypeptide sequence, but not a third amino acid residue).

By “c-terminally” or “c-terminal” to, it is meant toward the c-terminus. Therefore, by “c-terminally adjacent” it is meant “next to” and on the c-terminal side (i.e., on the right side if reading from left to right).

By “n-terminally” or “n-terminal” to, it is meant toward the n-terminus. Therefore, by “n-terminally adjacent” it is meant “next to” and on the n-terminal side (i.e., on the left side if reading from left to right).

As used herein, a “recombinant” or “engineered” cell refers to a cell into which an exogenous DNA sequence, such as a cDNA sequence, has been introduced. A “host cell” is one that contains such an exogenous DNA sequence. “Recombinant” as used herein to describe a polynucleotide means a polynucleotide which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide.

As used herein, “spontaneous” refers to a bond (e.g. an isopeptide or covalent bond) which forms in a protein or between polypeptides without the need for any other agent (e.g. an enzyme catalyst) to be present, and without chemical modification of the protein or peptide (e.g. without native chemical ligation or chemical coupling using 1-ethyl-3-(3-dimethylaminopropyl) carbodiimide (EDC)).

A “subject” is a living multi-cellular vertebrate organism. In the context of this disclosure, the subject can be an experimental subject, such as a non-human mammal, e.g., a mouse, a rat, or a non-human primate. Alternatively, the subject can be a human subject.

As used herein, “vector” refers to a vehicle by which nucleic acid molecules are contained and transferred from one environment to another or that facilitates the manipulation of a nucleic acid molecule. A vector may be, for example, a cloning vector, an expression vector, or a plasmid. Vectors include, for example, a BAC or a YAC vector. The term “expression vector” includes, without limitation, any vector, (e.g., a plasmid, cosmid or phage chromosome) containing a coding sequence suitable for expression by a cell (e.g., wherein the coding sequence is operatively linked to a transcriptional control element such as a promoter). A vector may comprise two or more nucleic acid molecules, in certain embodiments each of those two or more nucleic acid molecules comprises a nucleotide sequence that encodes a protein.

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is used herein to indicate a non-limiting example and is synonymous with the term “for example.”

It is further to be understood that all base sizes or amino acid sizes, and all molecular weight or molecular mass values, given for nucleic acid molecules or polypeptides are approximate and are provided for description. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specifically stated, a process comprising a step of mixing two or more components does not require any specific order of mixing. Thus components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not necessarily mean that the steps must be performed in that order (i.e., step 1 then step 2 then step 3, etc.). The word “then” may be used to specify the order of a method's steps.

The present invention is not limited to particular embodiments described herein. It is appreciated that certain features of the invention which are, for clarity, described in the context of separate embodiments, may also be provided in combination in a single embodiment. Conversely, various features of the invention which are, for brevity, described in the context of a single embodiment may also be provided separately or in any suitable sub-combination. All combinations of the embodiments are specifically embraced by the present invention and are disclosed herein just as if each and every combination was individually and explicitly disclosed. In addition, all sub-combinations are also specifically embraced by the present invention and are disclosed herein just as if each and every such sub-combination was individually and explicitly disclosed herein.

References, including patents and published patent applications, cited herein are incorporated by reference in their entirety.

Although methods and materials similar or equivalent to those described herein can be used in the practice or testing of this disclosure, suitable methods and materials are described below.

### EXAMPLES

### Example 1: GalacTag and GalacDock Polypeptides

GBS BP-1 (GBS80) contains isopeptide bonds in domain D2 (K210 and N351), and domain D3 (K387 and N515) (amino acid numbering based on SEQ ID NO: 40). GBS BP-2a (GBS59) contains isopeptide bonds in domain D2 (K199 and N325), domain D3 (K355 and N436), and domain D4 (K463 and N636) (amino acid numbering based on SEQ ID NO: 41). GBS BP-2b contains isopeptide bonds in domain D2 (K187 and N330), and domain D3 (K358 and N462) (amino acid numbering based on SEQ ID NO: 42).

The present inventors utilized sequences derived from domains D2-D3 of GBS BP-1 and BP2b, and domains D2-D4 of GBS BP-2a, to produce “GalacTag/GalacDock” pairs. Each pair has (1) a ‘GalacTag’ polypeptide sequence containing the isopeptide bond-forming asparagine, and (2) a ‘GalacDock’ polypeptide sequence containing the respective lysine partner. The minimum designed peptide length from the C-terminus of GBS BP pilus proteins for isopeptide formation is shown as a double dashed line for BP-1 (FIG. 1A), BP-2a (FIG. 1B), and BP-2b (FIG. 1C); the peptide length was extended (double solid line) to modulate solubility and stability.

Seven GalacTag/GalacDock polypeptides of different lengths were prepared based on GBS BP 1, BP 2a, and BP 2b proteins. The minimum length maintained the isopeptide bond and minimal beta sheet secondary structure contacts; the maximum length traversed the disordered loop before the next ordered beta-sheet.

GalacTags and GalacDocks 1-3 were derived from GBS BP 2b; GalacTags and GalacDocks 4 and 5 were derived from GBS BP 1; GalacTags and GalacDocks 6 and 7 were derived from GBS BP 2a. See Table 1.

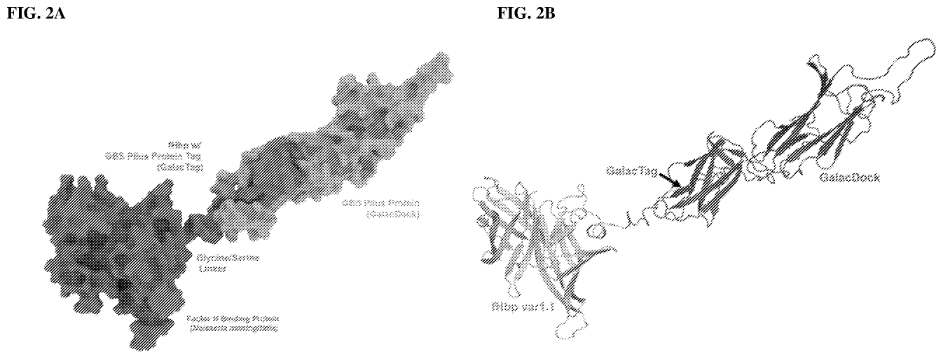

Additionally, a nucleotide sequence encoding each of the seven GalacTag polypeptides was linked to a nucleotide sequence encoding a target protein (factor H binding protein (fHbp) variant 1.1. from Neisseria meningitidis (SEQ ID NO:29) with a C-terminal 6xHistidine tag), to provide fusions of GalacTag peptides with the fHbp target moiety, linked by a five amino acid linker sequence and containing a C-terminal 6-histidine tag. See FIGS. 2A-2B, and Table 1.

TABLE 1

| | Derived from | SEQ ID NO |
|---|---|---|
| GalacTag1 | BP2b | SEQ ID NO: 8 |
| GalacTag2 | BP2b | SEQ ID NO: 9 |
| GalacTag3 | BP2b | SEQ ID NO: 10 |
| GalacTag4 | BP1 | SEQ ID NO: 11 |
| GalacTag5 | BP1 | SEQ ID NO: 12 |
| GalacTag6 | BP2a | SEQ ID NO: 13 |
| GalacTag7 | BP2a | SEQ ID NO: 14 |
| GalacDock1 | BP2b | SEQ ID NO: 1 |
| GalacDock2 | BP2b | SEQ ID NO: 2 |
| GalacDock3 | BP2b | SEQ ID NO: 3 |
| GalacDock4 | BP1 | SEQ ID NO: 4 |
| GalacDock5 | BP1 | SEQ ID NO: 5 |
| GalacDock6 | BP2a | SEQ ID NO: 6 |
| GalacDock7 | BP2a | SEQ ID NO: 7 |
| GalacTag1_fHbp | BP2b + fHbp | SEQ ID NO: 15 |
| GalacTag2_fHbp | BP2b + fHbp | SEQ ID NO: 16 |
| GalacTag3_fHbp | BP2b + fHbp | SEQ ID NO: 17 |
| GalacTag4_fHbp | BP1 + fHbp | SEQ ID NO: 18 |
| GalacTag5_fHbp | BP1 + fHbp | SEQ ID NO: 19 |
| GalacTag6_fHbp | BP2a + fHbp | SEQ ID NO: 20 |
| GalacTag7_fHbp | BP2a + fHbp | SEQ ID NO: 21 |

Nucleic acid sequences encoding each GalacDock and GalacTag+fHbp polypeptide, as shown in Table 1, were prepared and inserted into pET-28b(+) vectors. After cloning, each GalacDock and GalacTag+fHbp polypeptide was expressed in E. coli, BL21 (DE3) Star competent cells, using standard protocols. A single E. coli colony from each plate was inoculated in 5 mL Lennox Broth (LB) medium with Kanamycin for 6 hours and expanded to a 50 mL Magic auto-induction media (Thermo Fisher Scientific) with Kanamycin. After 24 hours, the cell culture was harvested by centrifuging at 4,000 rpm for minutes and then collecting the pellets.

Cell pellets were lysed using BugBuster lysis buffer and loaded onto an SDS-Page gel to detect protein expression and solubility of each GalacDock and GalacTag+fHbp polypeptide. Protein solubility was confirmed for GalacDock5, GalacDock6, and GalacDock7 polypeptides. Each of the seven GalacTag_fHbp proteins was at least partially soluble. Pellets from GalacDock5-7 and GalacTag5-7_fHbp were further purified using a HisTrap HP column.

### Example 2: Isopeptide-Driven Complex Formation of GalacDock and GalacTag

After purification using the HisTrap HP column, the ability of four GalacDock/GalacTag pairs to bind together was investigated (Table 2). Each GalacDock/GalacTag pair was mixed in vitro at a 1:1 molar ratio at room temperature for approximately 16 hours, and samples were then subjected to SDS-PAGE electrophoresis. Only one of the tested pairs (GalacDock6/GalacTag6_fHbp) formed a higher-order molecular weight complex.

TABLE 2

| GalacDock | GalacTag | |
|---|---|---|
| GalacDock5 | Galac Tag5_fHbp | No complex |
| (SEQ ID NO: 5 | (SEQ ID NO: 19) | |
| GalacDock6 | GalacTag6_fHbp | Complex |
| (SEQ ID NO: 6) | (SEQ ID NO: 20) | Forming |
| GalacDock7 | Galac Tag7_fHbp | No complex |
| (SEQ ID NO: 7) | (SEQ ID NO: 21) | |
| GalacDock6 | GalacTag7_fHbp | No complex |
| (SEQ ID NO: 6) | (SEQ ID NO: 21) | |

To assess whether the GalacDock6/GalacTag6_fHbp higher-order molecular weight complex was due to spontaneous isopeptide bond formation, three polypeptide variants of GlacDock6 (SEQ ID NO:6) were produced: one having the single amino acid substitution K463A (numbered according to BP2a reference SEQ ID NO: 41, corresponding to K282 in SEQ ID NO:6); one having a single amino acid substitution E589Q (BP2a reference SEQ ID NO: 41, corresponding to E408 in SEQ ID NO:6); and one having a single amino acid substitution E589A (BP2a reference SEQ ID NO: 41, corresponding to E408 in SEQ ID NO:6).

One polypeptide variant of GalacTag6_fHbp (SEQ ID NO:20) was produced, having single point substitution N636A in the GalacTag (numbered according to BP2a reference SEQ ID NO: 41, corresponding to N28 in SEQ ID NO:20).

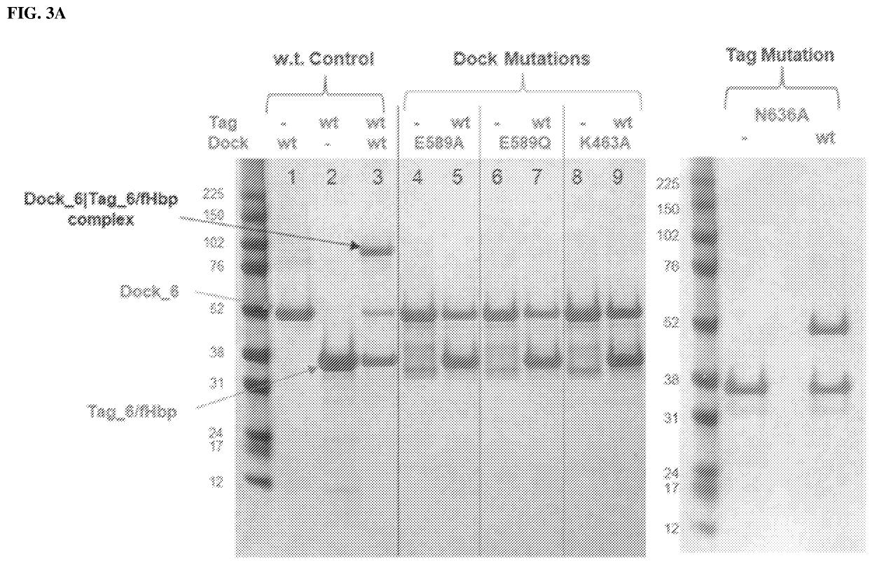

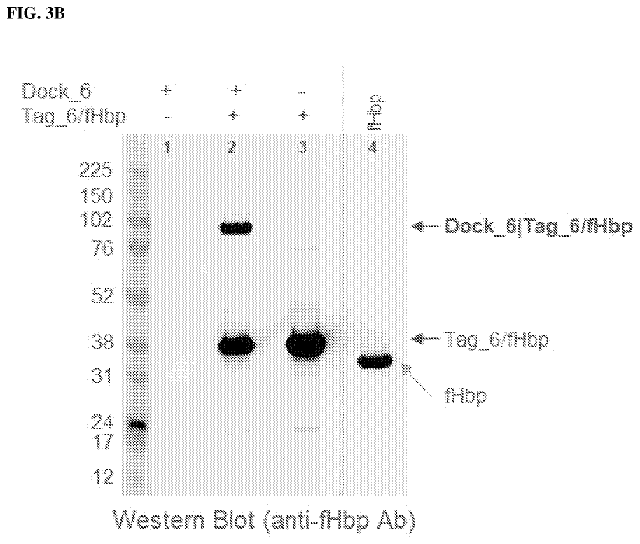

Using these variant GalacDock6 and GalacTag6_fHbp polypeptides, pairs as shown in Table 3 were mixed in vitro at a 1:1 molar ratio at room temperature for approximately 16 hours, and samples were then subjected to SDS-PAGE electrophoresis. As shown in FIG. 3A, complex formation was abolished when any of the variant GalacDoc6 polypeptides were tested with ‘wildtype’ (wt) GalacTag6_fHbp (SEQ ID NO:20). Complex formation was also abolished when the variant GalacTag6_fHbp (N636A) was tested with ‘wild type’ (wt) GalacDoc6 (SEQ ID NO:6). These results confirm that the individual lysine (K463), asparagine (N636), and glutamic acid (E589) residues in the GalacDock6/GalacTag6 binding pair drive the auto-catalytic isopeptide covalent bond formation.

TABLE 3

| | | Complex |
|---|---|---|
| GalacTag | GalacDoc | formation |
| none | wt | — |
| wt | none | — |
| wt | wt | yes |
| none | E589A | — |
| wt | E589A | no |
| none | E589Q | — |
| wt | E589Q | no |
| none | K463A | — |
| wt | K463A | no |
| N636A | none | — |
| N636A | wt | no |

Western blots were conducted on the GalacDock6/GalacTag6_ffibp higher-order molecular weight complex, using an anti-fHbp antibody (4B3). Results showed that the higher order species contained fHbp, which is attributed to complex formation by GalacTag6_ffibp (SEQ ID NO: 20) & GalacDock6 (SEQ ID NO:6) complex formation. (FIG. 3B).

### Example 3: Nanoparticle Complex

The use of the GalacTag6/GalacDock6 isopeptide system for the presentation of modular and complex biomacromolecules was assessed using nanoparticles composed of GBS ferritin polypeptide subunits.

Ferritin subunit polypeptides from GBS strains isolated from a human source (GBS DK-PW-092) and from a bovine source (GBS LMG Strain 14747, SEQ ID NO:30) were utilized. The ferritin subunit from GBS DK-PW-092 (GenBank KLL27267.1) contains a cysteine at residue 124 that, based on homology modeling, is not likely to form a disulfide bridge. A C124S substitution was made in the sequence to avoid potential aggregation (SEQ ID NO:31). Histidine tags (e.g., 6x-His tags) may be added to GBS ferritin subunits to aid in purification and may include a linker such as SEQ ID NO: 39.

The naturally-occurring S. pyogenes DPS-like peroxide resistance protein (Dpr) nanoparticle is made up of twelve identical protein subunits; each subunit contains an N-terminal helical portion. A similar helical sequence was not present in the GBS 092 or 14747 ferritin subunit polypeptides. Because the absence of an N-terminal helical portion could affect the colloidal stability and yield of multimeric particles, a chimeric molecule was created where the first (N-terminal) three amino acids of SEQ ID NO:31 (DK-PW-092 ferritin subunit) were replaced with the N-terminal 25 amino acids of S. pyogenes Dpr, to provide a chimeric polypeptide (SEQ ID NO:33, ‘092+helix’ or ‘092+N-terminal chimera’).

Polypeptide subunits of GBS ferritin nanoparticles were covalently linked (via glycine-serine residues) to C-terminus of GalacTag6 peptide (SEQ ID NO:13) by recombinant expression. Three different GBS Ferritin NP subunits were used: GBS LMG 14747 (SEQ ID NO: 30), GBS DK-PW-092 (SEQ ID NO: 31), and GBS DK-PW-092+helix (SEQ ID NO:33). Separate cloning vectors were designed for each of the three GalacTag6_GBSFerritinSubunit constructs.

The GalacTag6_GBSFerritin constructs were expressed in Terrific Broth (TB) medium using fast Isopropyl β-d-l-thiogalactopyranoside (IPTG) induction. Cell pellets were collected by centrifugation at 4,000 rpm for 20 minutes. A small amount of pellet from each GalacTag6_GBSFerritin construct was lysed using BugBuster lysis buffer and run on SDS-Page gels to confirm expression and solubility.

The remaining pellets were resuspended in lysis buffer (BugBuster+Buffer A for Nickel NTA purification) on a rocker at room temperature for 30 minutes, then sonicated for 5 minutes to lyse the protein. Supernatant was collected after centrifugation at 14,000 rpm for 20 minutes and run over two 5 mL HisTrap HP columns. Afterwards, size-exclusion chromatography was run on the his-tag purified sample on a Superose 6 Increase 10/300 GL column with 10 mM HEPES pH 7.5, 300 mM NaCl, 5% Glycerol buffer.

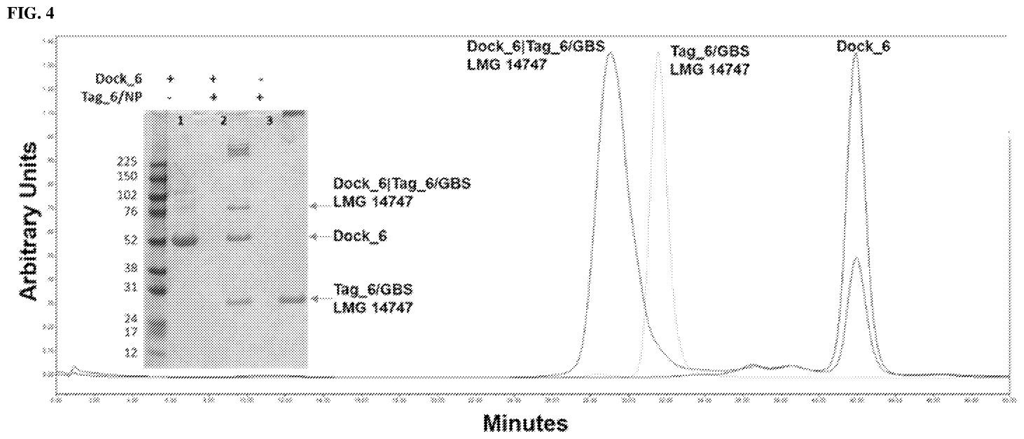

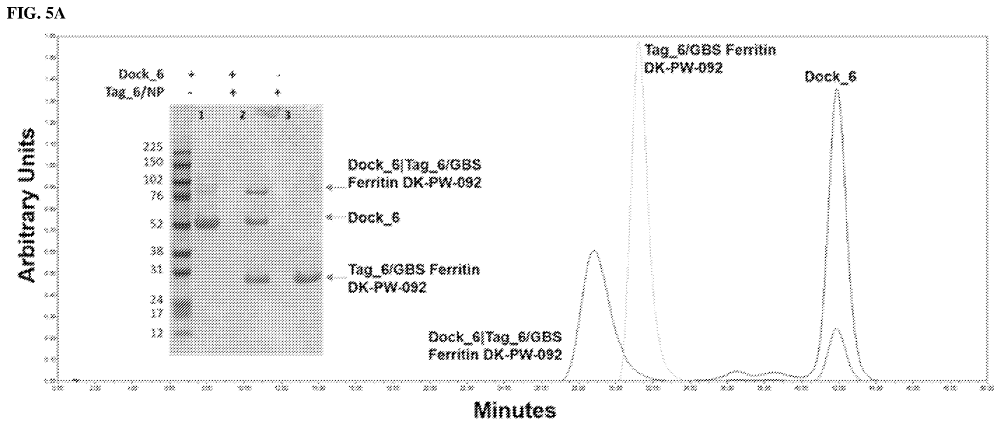

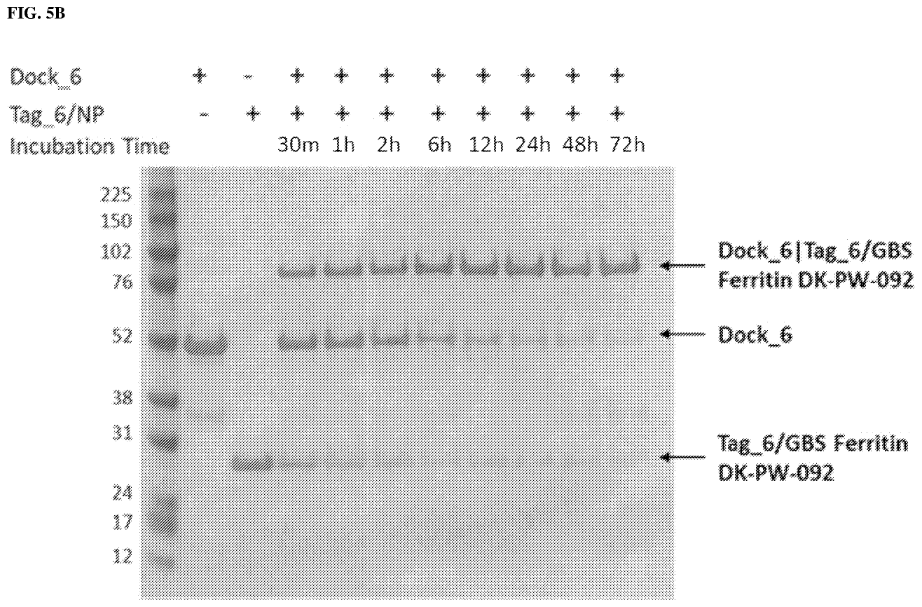

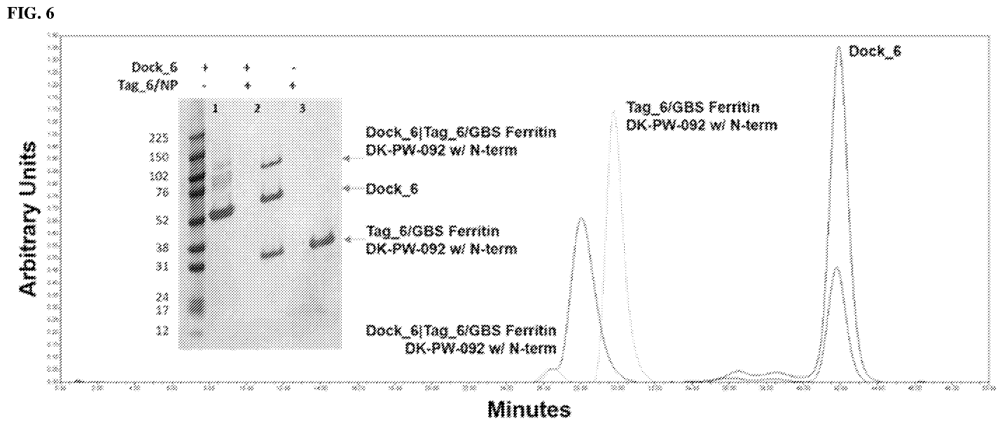

The GBS ferritin nanoparticles made of subunits that were covalently linked to GalacTag6 were then in vitro mixed at a 1:1 ratio with GalacDock6 (SEQ ID NO:6) at room temperature for approximately 16 hours, yielding higher-order oligomer complexes, as shown by both SDS-Page gel and HPLC SEC (FIGS. 4, 5A and 6). Complex forming of GalacDock6/GalacTag6/GBS Ferritin DK-PW-092 was observed starting at t=30 minutes and increased in a time-dependent manner from 30 minutes to 72 hours (FIG. 5B).

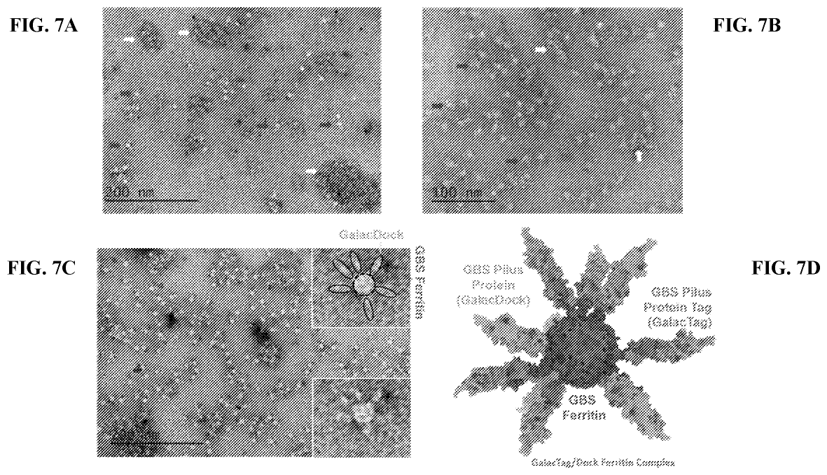

To confirm the presence of GBS nanoparticles covalently linked to GalacTag6, negative stain electron microscopy (NS-EM) was performed to visualize the morphology of the nanoparticles (FIGS. 7A-7C). Purified complex samples of each of the three types of GalacDock6_GalacTag6_GBSFerritin nanoparticles were pipetted onto NS-EM grids. The NS-EM grids were prepared by applying each sample separately onto a carbon coated copper grid, followed by staining with Methylamine Tungstate. The grids were then screened using a JEOL JEM-1230 transmission electron microscope. GalacDock_GalacTag6_GBSFerritinDK-PW-092 complexed samples had the most uniform morphology, and displayed spherical particles arrayed with tubular proteins (FIG. 7C), reasonably corresponding to twelve GalacDock/GalacTag polypeptides repetitively arrayed on a dodecameric GBS ferritin nanoparticle, as modeled in FIG. 7D.

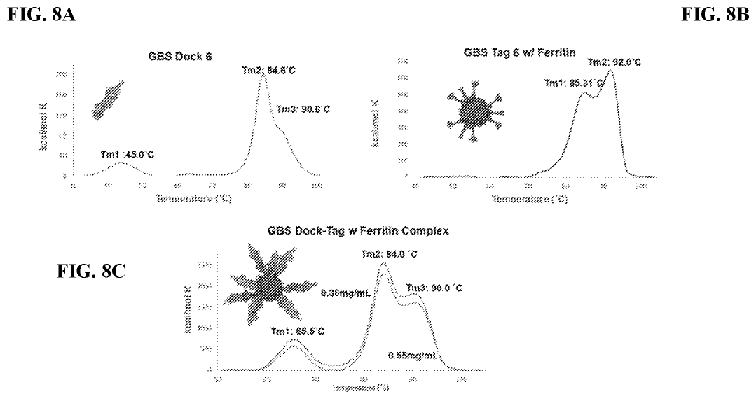

To gain an understanding of the thermodynamic properties of the GalacTag_nanoparticle_GalacDock complexes, purified samples of GalacDock6, GalacTag6_GBSFerritinDK-PW-092, and GalacDock_GalacTag6_GBSFerritinDK-PW-092 were subjected to differential scanning calorimetry (Malvern NanoDSC). The thermal analysis was run at two concentrations (0.36 mg/mL and 0.55 mg/mL), relative to uncomplexed samples of GalacDock6 and GalacTag6_GBSFerritinDK-PW-092 nanoparticles. For GalacDock6, the DSC thermogram (FIG. 8A) indicates the presence of three thermal unfolding transitions (potentially corresponding to the three domains D2-D4 in GBS BP-2a). The DSC thermogram of GalacTag6_GBSFerritinDK-PW-092 (FIG. 8B), indicates the presence of two thermal unfolding transitions. The DSC thermogram for GalacDock6_GalacTag6_GBSFerritinDK-PW-092 nanoparticles (FIG. 8C) indicates the presence of three thermal unfolding transitions, with the apparent stabilization of Tm₁ (65.5° C.) relative to Tm₁ (45° C.) in uncomplexed GalacDock6. These results indicate that complex formation increases the stability for this unfolding event.

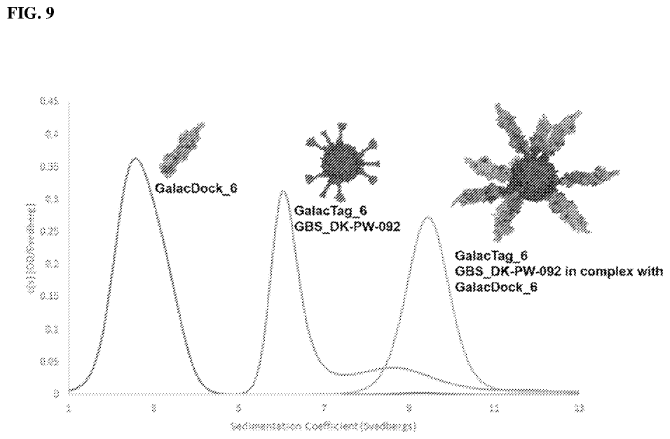

Analytical ultracentrifugation (AUC) was performed on (a) GalacDock6, (b) GalacTag6_GBSFerritinDK-PW-092, and (c) GalacDock6_GalacTag6_GB SFerritinDK-PW-092. The sedimentation coefficient, which relates to size, was calculated on the different proteins, revealing three distinct non-aggregated species, correlating with the HPLC-SEC and NS-EM experiments (FIG. 9).

### Example 4: GalacTag and GalacDock Polypeptides Derived from AP-1

GBS AP1 of PI-1 (AP1-1) contains isopeptide bonds in domain D4 (K740 and N850; amino acid numbering based on the AP1-1 polypeptide reference sequence of SEQ ID NO: 61). GBS AP1 of PI-2a (AP1-2a) contains isopeptide bonds in domain D4 (K742 and N850); amino acid numbering based on SEQ ID NO: 62).

The present inventors utilized sequences derived from domain D4 of GBS AP1-1 and AP1-2a to design “GalacTag/GalacDock” pairs. Each pair has (1) a ‘GalacTag’ polypeptide sequence containing the isopeptide bond-forming asparagine, and (2) a ‘GalacDock’ polypeptide sequence containing the respective lysine partner.

GalacTag8 and GalacDock8 are derived from GBS AP1-1; GalacTag9 and GalacDock9 are derived from GBS AP1-2a. See Table 2.

A nucleotide sequence encoding each of GalacTag polypeptides is linked to a nucleotide sequence encoding a target protein (e.g., factor H binding protein (fHbp) variant 1.1. from Neisseria meningitidis (SEQ ID NO:29) with a C-terminal 6xHistidine tag)), to provide fusions of GalacTag peptides with the target moiety.

TABLE 2

| | Derived from | SEQ ID NO |
|---|---|---|
| GalacTag8 | AP1-1 | SEQ ID NO: 50 |
| GalacDock8 | AP1-1 | SEQ ID NO: 59 |
| GalacTag9 | AP1-2a | SEQ ID NO: 51 |
| GalacDock9 | AP1-2a | SEQ ID NO: 60 |

1. A polypeptide comprising of an amino acid sequence selected from: SEQ ID NO: 1, SEQ ID NO: 2, SEQ ID NO: 3, SEQ ID NO: 4, SEQ ID NO:5, SEQ ID NO: 6, SEQ ID NO: 7, SEQ ID NO: 43, SEQ ID NO: 44, SEQ ID NO: 45, SEQ ID NO: 46, SEQ ID NO: 47, SEQ ID NO: 48, SEQ ID NO: 49, SEQ ID NO: 50, and SEQ ID NO: 51.

2. The polypeptide according to claim 1, comprising of amino acids 1-419 of SEQ ID NO:28.

3. The polypeptide according to claim 1, comprising of a sequence having at least 90%, at least 92%, at least 95%, at least 97%, at least 98%, or at least 99% identity to amino acids 1-419 of SEQ ID NO:28.

4. A fusion protein comprising a polypeptide according to claim 1 and a heterologous polypeptide, wherein (a) the heterologous polypeptide is covalently linked to the N-terminal amino acid of the polypeptide, either directly or via an amino acid linker; or (b) the heterologous polypeptide is covalently linked to the C-terminal amino acid of the polypeptide, either directly or via an amino acid linker.

5-6. (canceled)

7. The fusion protein of claim 4, wherein the heterologous polypeptide is an antigenic polypeptide.

8. The fusion protein of claim 4, wherein the heterologous antigenic polypeptide is an antigenic GBS surface protein or immunogenic fragment thereof.

9. The polypeptide of claim 1 conjugated to a bacterial capsular polysaccharide, wherein the bacterial capsular polysaccharide is also conjugated to a carrier protein.

10-11. (canceled)

12. A polypeptide comprising of an amino acid sequence selected from: SEQ ID NO: 8, SEQ ID NO:9, SEQ ID NO: 10, SEQ ID NO: 11, SEQ ID NO: 12, SEQ ID NO: 13, SEQ ID NO: 15, SEQ ID NO: 52, SEQ ID NO: 53, SEQ ID NO: 54, SEQ ID NO: 55, SEQ ID NO: 56, SEQ ID NO: 57, SEQ ID NO: 58, SEQ ID NO: 59, and SEQ ID NO: 60.

13. The polypeptide of claim 12, comprising of amino acids 422-452 of SEQ ID NO: 28.

14. The polypeptide of claim 12, comprising of a sequence having at least 90%, at least 93%, or at least 96% identity to amino acids 422-452 of SEQ ID NO: 28.

15. A fusion protein comprising the polypeptide of claim 12 and a heterologous polypeptide, wherein the heterologous polypeptide is a polypeptide subunit of a self-assembling protein nanoparticle.

16-24. (canceled)

25. The fusion protein of claim 15, wherein the polypeptide subunit of a self-assembling protein nanoparticle is a GBS ferritin polypeptide subunit, and wherein the polypeptide subunit is selected from a sequence comprising of SEQ ID NO: 30, SEQ ID NO: 31, SEQ ID NO: 32, and SEQ ID NO: 33.

26-40. (canceled)

41. A nucleic acid molecule encoding the polypeptide of claim 1.

42. A vector comprising the nucleic acid molecule of claim 41.

43-45. (canceled)

46. A method of producing a protein nanoparticle (NP) displaying a peptide tag on the NP exterior surface, where said peptide tag is a fragment of an isopeptide protein, said tag having a length of at least 5 amino acids but no more than 50 amino acids, and comprising a first reactive residue involved in formation of an intramolecular isopeptide bond in said isopeptide protein, and wherein said isopeptide protein is a Group B Streptococcus (GBS) pilus protein selected from the group consisting of (i) a Group B Streptococcus (GBS) pilus Backbone Protein (BP) or a protein with at least 95% identity thereto; and (ii) a Group B Streptococcus (GBS) pilus Ancillary Protein (AP) or a protein with at least 95% identity thereto; and wherein said isopeptide protein is and capable of spontaneously forming an isopeptide bond, said method comprising:
(a) recombinantly expressing fusion proteins of said peptide tag and a NP polypeptide subunit, in a host cell under conditions that allow self-assembly of said nanoparticle subunits into a NP; and
(b) isolating or purifying the NP.

47-48. (canceled)

49. A method of producing the fusion protein of claim 4 comprising:
(a) providing a peptide tag that is a fragment of an isopeptide protein, said tag having a length of at least 5 amino acids but no more than 50 amino acids, and comprising a first reactive residue involved in formation of an intramolecular isopeptide bond in said isopeptide protein, wherein said peptide tag is either unconjugated or is conjugated to a heterologous polypeptide or to another molecule, and wherein said isopeptide protein is a Group B Streptococcus (GBS) pilus Backbone Protein (BP) or Ancillary Protein (AP);
(b) providing a peptide binding partner to said peptide tag, where said binding partner comprises a different fragment of said isopeptide protein, wherein said fragment is at least 20 amino acids in length and comprises a second reactive residue involved in said isopeptide bond in said isopeptide protein, wherein the binding partner does not include the first reactive residue of the peptide tag; and
(c) contacting said peptide tag and binding partner under conditions that allow the peptide tag and binding partner to form an isopeptide bond between the first and second reactive residues,
wherein at least one of said peptide tag and binding partner is covalently attached to a heterologous molecule.

50-68. (canceled)

69. A pharmaceutical composition comprising a fusion protein according to claim 15.

70-73. (canceled)

74. A method of inducing an immune response in a subject, comprising administering to the subject an immunologically effective amount of a polypeptide according to claim 1.


SEQUENCE LISTING (63 sequences)

SEQ ID NO: 1 - protein, 266 aa, Artificial Sequence
  source: /note=“Description of Artificial Sequence Synthetic polypeptide”
MHHHHHHSGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILG

SEQ ID NO: 2 - protein, 274 aa, Artificial Sequence
  source: /note=“Description of Artificial Sequence Synthetic polypeptide”
MHHHHHHSGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTN

SEQ ID NO: 3 - protein, 281 aa, Artificial Sequence
  source: /note=“Description of Artificial Sequence Synthetic polypeptide”
MHHHHHHSGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVN

SEQ ID NO: 4 - protein, 299 aa, Artificial Sequence
  source: /note=“Description of Artificial Sequence Synthetic polypeptide”
MHHHHHHSNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVS

SEQ ID NO: 5 - protein, 316 aa, Artificial Sequence
  source: /note=“Description of Artificial Sequence Synthetic polypeptide”
MHHHHHHSNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSAD

SEQ ID NO: 6 - protein, 427 aa, Artificial Sequence
  source: /note=“Description of Artificial Sequence Synthetic polypeptide”
MHHHHHHSNTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVN

SEQ ID NO: 7 - protein, 450 aa, Artificial Sequence
  source: /note=“Description of Artificial Sequence Synthetic polypeptide”
MHHHHHHSNTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVNATSYSKGSAQDIEYTQGSKTKDA

SEQ ID NO: 8 - protein, 28 aa, Artificial Sequence
  source: /note=“Description of Artificial Sequence Synthetic peptide”
MGATDTTNSDNLLVNPTVENNKGTELPS

SEQ ID NO: 9 - protein, 19 aa, Artificial Sequence
  source: /note=“Description of Artificial Sequence Synthetic peptide”
MALLVNPTVEDNKGTELPS

SEQ ID NO: 10 - protein, 12 aa, Artificial Sequence
  source: /note=“Description of Artificial Sequence Synthetic peptide”
MAENNKGTELPS

SEQ ID NO: 11 - protein, 28 aa, Artificial Sequence
  source: /note=“Description of Artificial Sequence Synthetic peptide”
MASYNTKPTDITVDSADATPDTIKNNKR

SEQ ID NO: 12 - protein, 10 aa, Artificial Sequence
  source: /note=“Description of Artificial Sequence Synthetic peptide”
MADTIKDNKR

SEQ ID NO: 13 - protein, 33 aa, Artificial Sequence
  source: /note=“Description of Artificial Sequence Synthetic polypeptide”
MASYSKGSAQDIEYTQGSKTKDAQQVINKKVTI

SEQ ID NO: 14 - protein, 10 aa, Artificial Sequence
  source: /note=“Description of Artificial Sequence Synthetic peptide”
MAVINKKVTI

SEQ ID NO: 15 - protein, 285 aa, Artificial Sequence
  source: /note=“Description of Artificial Sequence Synthetic polypeptide”
MGATDTTNSDNLLVNPTVENNKGTELPSGGSGGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGSHHHHHH

SEQ ID NO: 16 - protein, 276 aa, Artificial Sequence
  source: /note=“Description of Artificial Sequence Synthetic polypeptide”
MALLVNPTVENNKGTELPSGGSGGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGSHHHHHH

SEQ ID NO: 17 - protein, 269 aa, Artificial Sequence
  source: /note=“Description of Artificial Sequence Synthetic polypeptide”
MAENNKGTELPSGGSGGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGSHHHHHH

SEQ ID NO: 18 - protein, 285 aa, Artificial Sequence
  source: /note=“Description of Artificial Sequence Synthetic polypeptide”
MASYNTKPTDITVDSADATPDTIKNNKRGGSGGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGSHHHHHH

SEQ ID NO: 19 - protein, 267 aa, Artificial Sequence
  source: /note=“Description of Artificial Sequence Synthetic polypeptide”
MADTIKNNKRGGSGGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGSHHHHHH

SEQ ID NO: 20 - protein, 290 aa, Artificial Sequence
  source: /note=“Description of Artificial Sequence Synthetic polypeptide”
MASYSKGSAQDIEYTQGSKTKDAQQVINKKVTIGGSGGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGSHHHHHH

SEQ ID NO: 21 - protein, 267 aa, Artificial Sequence
  source: /note=“Description of Artificial Sequence Synthetic polypeptide”
MAVINKKVTIGGSGGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGSHHHHHH

SEQ ID NO: 22 - protein, 199 aa, Artificial Sequence
  source: /note=“Description of Artificial Sequence Synthetic polypeptide”
MASYSKGSAQDIEYTQGSKTKDAQQVINKKVTIGGSGVYEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGLGSSGHHHHHH

SEQ ID NO: 23 - protein, 201 aa, Artificial Sequence
  source: /note=“Description of Artificial Sequence Synthetic polypeptide”
MASYSKGSAQDIEYTQGSKTKDAQQVINKKVTIGGSGKFEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDAGSSGHHHHHH

SEQ ID NO: 24 - protein, 221 aa, Artificial Sequence
  source: /note=“Description of Artificial Sequence Synthetic polypeptide”
MASYSKGSAQDIEYTQGSKTKDAQQVINKKVTIGGSGTNTLVENIYASVTHNISKKEASKNEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGLGSSGHHHHHH

SEQ ID NO: 25 - protein, 223 aa, Artificial Sequence
  source: /note=“Description of Artificial Sequence Synthetic polypeptide”
MASYSKGSAQDIEYTQGSKTKDAQQVINKKVTIGGSGTNTLVENIYASVTHNISKKEASKNEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDAGSSGHHHHHH

SEQ ID NO: 26 - protein, 292 aa, Streptococcus sp.
MGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSHHHHH

SEQ ID NO: 27 - protein, 319 aa, Streptococcus sp.
NVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKR

SEQ ID NO: 28 - protein, 452 aa, Streptococcus sp.
NTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVNATSYSKGSAQDIEYTQGSKTKDAQQVINKKVTI

SEQ ID NO: 29 - protein, 246 aa, Artificial Sequence
  source: /note=“Description of Artificial Sequence Synthetic polypeptide”
AGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQLGGS

SEQ ID NO: 30 - protein, 153 aa, Streptococcus sp.
MVYEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGL

SEQ ID NO: 31 - protein, 155 aa, Streptococcus agalactiae
MKFEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDA

SEQ ID NO: 32 - protein, 175 aa, Artificial Sequence
  source: /note=“Description of Artificial Sequence Synthetic polypeptide”
MTNTLVENIYASVTHNISKKEASKNEKTQAVLNQAVADLSKASSIVHQTHWYMRGPGFFYLHPKMDELMDELNETLDVVSERLITIGGAPYSTLKEFDEHSKLTEEVGSYDKSMTERLEKLADVYAYLSDLFQEGLDVSDEEGDDPSNGIFSDAQDAVQKTLWMLQAEIGRSSGL

SEQ ID NO: 33 - protein, 177 aa, Artificial Sequence
  source: /note=“Description of Artificial Sequence Synthetic polypeptide”
MTNTLVENIYASVTHNISKKEASKNEKTKEILNQLVADLSQFSVVIHQTHWYMRGPEFLTLHPQMDEYMDQINEQLDVVSERLITLDGSPFSTLREFAENTKIEDEIGNWDRTIPERMEKLVAGYRYLADLYAKGIEVSGEEGDDSTQDIFIANKTDIEKNIWMLQAKLGKAPGIDA

SEQ ID NO: 34 - protein, 6 aa, Artificial Sequence
  source: /note=“Description of Artificial Sequence Synthetic 6xHis tag”
HHHHHH

SEQ ID NO: 35 - protein, 8 aa, Artificial Sequence
  source: /note=“Description of Artificial Sequence Synthetic peptide”
DYKDDDDK

SEQ ID NO: 36 - protein, 9 aa, Artificial Sequence
  source: /note=“Description of Artificial Sequence Synthetic peptide”
AWRHPQFGG

SEQ ID NO: 37 - protein, 8 aa, Artificial Sequence
  source: /note=“Description of Artificial Sequence Synthetic peptide”
WSHPQFEK

SEQ ID NO: 38 - protein, 28 aa, Artificial Sequence
  source: /note=“Description of Artificial Sequence Synthetic peptide”
WSHPQFEKGGGSGGGSGGGSWSHPQFEK

SEQ ID NO: 39 - protein, 5 aa, Artificial Sequence
  source: /note=“Description of Artificial Sequence Synthetic peptide”
GGSGG

SEQ ID NO: 40 - protein, 554 aa, Streptococcus sp.
MKLSKKLLFSAAVLTMVAGSTVEPVAQFATGMSIVRAAEVSQERPAKTTVNIYKLQADSYKSEITSNGGIENKDGEVISNYAKLGDNVKGLQGVQFKRYKVKTDISVDELKKLTTVEAADAKVGTILEEGVSLPQKTNAQGLVVDALDSKSNVRYLYVEDLKNSPSNITKAYAVPFVLELPVANSTGTGFLSEINIYPKNVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSADATPDTIKNNKRPSIPNTGGIGTAIFVAIGAAVMAFAVKGMKRRTKDN

SEQ ID NO: 41 - protein, 675 aa, Streptococcus sp.
MKKINKYFAVFSALLLTVTSLFSVAPVFAEEAKTTDTVTLHKIVMPRTAFDGFTAGTKGKDNIDYVGKQIEDLKTYFGSGEAKEIAGAYFAFKNEAGTKYITENGEEVDTLDTTDAKGCAVLKGLTTDNGFKFNTSKLTGTYQIVELKEKSTYNNDGSILADSKAVPVKITLPLVNDNGVVKDAHVYPKNTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVNATSYSKGSAQDIEYTQGSKTKDAQQVINKKVTIPQTGGIGTIFFTIIGLSIMLGAVVIMKRRQSEEV

SEQ ID NO: 42 - protein, 502 aa, Streptococcus sp.
MKKKMIQSLLVASLAFGMAVSPVTPIAFAAETGTITVQDTQKGATYKAYKVFDAEIDNANVSDSNKDGASYLIPQGKEAEYKASTDFNSLFTTTTNGGRTYVTKKDTASANEIATWAKSISANTTPVSTVTESNNDGTEVINVSQYGYYYVSSTVNNGAVIMVTSVTPNATIHEKNTDATWGDGGGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVNPTVENNKGTELPSTGGIGTTIFYIIGAILVIGAGIVLVARRRLRS

SEQ ID NO: 43 - protein, 258 aa, Streptococcus sp.
GGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILG

SEQ ID NO: 44 - protein, 266 aa, Streptococcus sp.
GGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTN

SEQ ID NO: 45 - protein, 273 aa, Streptococcus sp.
GGKTVDQKTYSVGDTVKYTITYKNAVNYHGTEKVYQYVIKDTMPSASVVDLNEGSYEVTITDGSGNITTLTQGSEKATGKYNLLEENNNFTITIPWAATNTPTGNTQNGANDDFFYKGINTITVTYTGVLKSGAKPGSADLPENTNIATINPNTSNDDPGQKVTVRDGQITIKKIDGSTKASLQGAIFVLKNATGQFLNFNDTNNVEWGTEANATEYTTGADGIITITGLKEGTYYLVEKKAPLGYNLLDNSQKVILGDGATDTTNSDNLLVN

SEQ ID NO: 46 - protein, 291 aa, Streptococcus sp.
NVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVS

SEQ ID NO: 47 - protein, 308 aa, Streptococcus sp.
NVVTDEPKTDKDVKKLGQDDAGYTIGEEFKWFLKSTIPANLGDYEKFEITDKFADGLTYKSVGKIKIGSKTLNRDEHYTIDEPTVDNQNTLKITFKPEKFKEIAELLKGMTLVKNQDALDKATANTDDAAFLEIPVASTINEKAVLGKAIENTFELQYDHTPDKADNPKPSNPPRKPEVHTGGKRFVKKDSTETQTLGGAEFDLLASDGTAVKWTDALIKANTNKNYIAGEAVTGQPIKLKSHTDGTFEIKGLAYAVDANAEGTAVTYKLKETKAPEGYVIPDKEIEFTVSQTSYNTKPTDITVDSAD

SEQ ID NO: 48 - protein, 419 aa, Streptococcus sp.
NTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVN

SEQ ID NO: 49 - protein, 442 aa, Streptococcus sp.
NTETKPQVDKNFADKELDYANNKKDKGTVSASVGDVKKYHVGTKILKGSDYKKLIWTDSMTKGLTFNNDIAVTLDGATLDATNYKLVADDQGFRLVLTDKGLEAVAKAAKTKDVEIKITYSATLNGSAVVEVLETNDVKLDYGNNPTIENEPKEGIPVDKKITVNKTWAVDGNEVNKADETVDAVFTLQVKDGDKWVNVDSAKATAATSFKHTFENLDNAKTYRVIERVSGYAPEYVSFVNGVVTIKNNKDSNEPTPINPSEPKVVTYGRKFVKTNKDGKERLAGATFLVKKDGKYLARKSGVATDAEKAAVDSTKSALDAAVKAYNDLTKEKQEGQDGKSALATVSEKQKAYNDAFVKANYSYEWVEDKNAKNVVKLISNDKGQFEITGLTEGQYSLEETQAPTGYAKLSGDVSFNVNATSYSKGSAQDIEYTQGSKTKDA

SEQ ID NO: 50 - protein, 114 aa, Streptococcus sp.
DKHSESLLGAKFQLQIEKDFSGYKQFVPEGSDVTTKNDGKIYFKALQDGNYKLYEISSPDGYIEVKTKPVVTFTIQNGEVTNLKADPNANKNQIGYLEGNGKHLITNTPKRPPG

SEQ ID NO: 51 - protein, 112 aa, Streptococcus sp.
DNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKG

SEQ ID NO: 52 - protein, 27 aa, Streptococcus sp.
GATDTTNSDNLLVNPTVENNKGTELPS

SEQ ID NO: 53 - protein, 18 aa, Streptococcus sp.
ALLVNPTVENNKGTELPS

SEQ ID NO: 54 - protein, 12 aa, Streptococcus sp.
MAENNKGTELPS

SEQ ID NO: 55 - protein, 28 aa, Streptococcus sp.
MASYNTKPTDITVDSADATPDTIKNNKR

SEQ ID NO: 56 - protein, 9 aa, Streptococcus sp.
ADTIKNNKR

SEQ ID NO: 57 - protein, 33 aa, Streptococcus sp.; identical to SEQ ID NO: 13

SEQ ID NO: 58 - protein, 10 aa, Streptococcus sp.
MAVINKKVTI

SEQ ID NO: 59 - protein, 12 aa, Streptococcus sp.
KMGEVEFIKVNK

SEQ ID NO: 60 - protein, 16 aa, Streptococcus sp.
KNEKKLGEIEFIKVDK

SEQ ID NO: 61 - protein, 890 aa, Streptococcus sp.
MKKRQKIWRGLSVTLLILSQIPFGILVQGETQDTNQALGKVIVKKTGDNATPLGKATFVLKNDNDKSETSHETVEGSGEATFENIKPGDYTLREETAPIGYKKTDKTWKVKVADNGATIIEGMDADKAEKRKEVLNAQYPKSAIYEDTKENYPLVNVEGSKVGEQYKALNPINGKDGRREIAEGWLSKKITGVNDLDKNKYKIELTVEGKTTVETKELNQPLDVVVLLDNSNSMNNERANNSQRALKAGEAVEKLIDKITSNKDNRVALVTYASTIFDGTEATVSKGVADQNGKALNDSVSWDYHKTTFTATTHNYSYLNLTNDANEVNILKSRIPKEAEHINGDRTLYQFGATFTQKALMKANEILETQSSNARKKLIFHVTDGVPTMSYAINFNPYISTSYQNQFNSFLNKIPDRSGILQEDFIINGDDYQIVKGDGESFKLFSDRKVPVTGGTTQAAYRVPQNQLSVMSNEGYAINSGYIYLYWRDYNWVYPFDPKTKKVSATKQIKTHGEPTTLYFNGNIRPKGYDIFTVGIGVNGDPGATPLEAEKFMQSISSKTENYTNVDDTNKIYDELNKYFKTIVEEKHSIVDGNVTDPMGEMIEFQLKNGQSFTHDDYVLVGNDGSQLKNGVALGGPNSDGGILKDVTVTYDKTSQTIKINHLNLGSGQKVVLTYDVRLKDNYISNKFYNTNNRTTLSPKSEKEPNTIRDFPIPKIRDVREFPVLTISNQKKMGEVEFIKVNKDKHSESLLGAKFQLQIEKDFSGYKQFVPEGSDVTTKNDGKIYFKALQDGNYKLYEISSPDGYIEVKTKPVVTFTIQNGEVTNLKADPNANKNQIGYLEGNGKHLITNTPKRPPGVFPKTGGIGTIVYILVGSTFMILTICSFRRKQL

SEQ ID NO: 62 - protein, 901 aa, Streptococcus sp.
MRKYQKFSKILTLSLFCLSQIPLNTNVLGESTVPENGAKGKLVVKKTDDQNKPLSKATFVLKTTAHPESKIEKVTAELTGEATFDNLIPGDYTLSEETAPEGYKKTNQTWQVKVESNGKTTIQNSGDKNSTIGQNQEELDKQYPPTGIYEDTKESYKLEHVKGSVPNGKSEAKAVNPYSSEGEHIREIPEGTLSKRISEVGDLAHNKYKIELTVSGKTIVKPVDKQKPLDVVFVLDNSNSMNNDGPNFQRHNKAKKAAEALGTAVKDILGANSDNRVALVTYGSDIFDGRSVDVVKGFKEDDKYYGLQTKFTIQTENYSHKQLTNNAEEIIKRIPTEAPKAKWGSTTNGLTPEQQKEYYLSKVGETFTMKAFMEADDILSQVNRNSQKIIVHVTDGVPTRSYAINNFKLGASYESQFEQMKKNGYLNKSNFLLTDKPEDIKGNGESYFLFPLDSYQTQIISGNLQKLHYLDLNLNYPKGTIYRNGPVKEHGTPTKLYINSLKQKNYDIFNFGIDISGFRQVYNEEYKKNQDGTFQKLKEEAFKLSDGEITELMRSFSSKPEYYTPIVTSADTSNNEILSKIQQQFETILTKENSIVNGTIEDPMGDKINLQLGNGQTLQPSDYTLQGNDGSVMKDGIATGGPNNDGGILKGVKLEYIGNKLYVRGLNLGEGQKVTLTYDVKLDDSFISNKFYDTNGRTTLNPKSEDPNTLRDFPIPKIRDVREYPTITIKNEKKLGEIEFIKVDKDNNKLLLKGATFELQEFNEDYKLYLPIKNNNSKVVTGENGKISYKDLKDGKYQLIEAVSPEDYQKITNKPILTFEVVKGSIKNIIAVNKQISEYHEEGDKHLITNTHIPPKGIIPMTGGKGILSFILIGGAMMSIAGGIYIWKRYKKSSDMSIKKD

SEQ ID NO: 63 - protein, 5 aa, Artificial Sequence
  source: /note=“Description of Artificial Sequence Synthetic peptide”
GSGGG